(19) United States
(12) Patent Application Publication
(10) Pub. No.: US 2015/0216136 A1
(43) Pub. Date: Aug. 6, 2015

(54) PLANT REGENERATION FROM PROTOPLASTS DERIVED FROM ELAEIS sp SUSPENSION CULTURES

(71) Applicants: MALAYSIAN PALM OIL BOARD, Kajang, Selangor (MY); FRAUNHOFER INSTITUTE FOR MOLECULAR BIOLOGY AND ECOLOGY (IME), Munchen (DE)

(72) Inventors: Abdul Masani Mat Yunus, Kajang (MY); Gundula Noll, Munster (DE); Ahmad Parveez Ghulam Kadir, Kajang (MY); Dirk Prufer, Munster (DE); Ravigadevi Sambanthamurthi, Kajang (MY)

(21) Appl. No.: 14/422,172

(22) PCT Filed: Aug. 7, 2013
(86) PCT No.: PCT/MY2013/000145

§ 371 Date: Feb. 17, 2015

(30) Foreign Application Priority Data
Aug. 15, 2012 (MY) PI 2012700558

(51) Int. Cl. A01H 4/00; C12N 15/82

(52) U.S. Cl. CPC A01H 4/008; C12N 15/8206

(57) ABSTRACT

The present disclosure relates to a protocol for the regeneration of Elaeis plants from protoplasts and to the genetic manipulation of the protoplasts to introduce or facilitate expression of desired properties and beneficial traits in Elaeis plants.

### FIELD OF INVENTION

The present disclosure relates to a protocol for the regeneration of Elaeis plants from protoplasts and to the genetic manipulation of the protoplasts to introduce or facilitate expression of desired properties and beneficial traits in Elaeis plants.

### BACKGROUND OF THE INVENTION

Bibliographic details of the publications referred to by author in this specification are collected alphabetically at the end of the description.

Reference to any prior art in this specification is not, and should not be taken as, an acknowledgment or any form of suggestion that this prior art forms part of the common general knowledge in any country.

The demand for oils and fats is dramatically increasing with a concomitant need for sustainable resources. Oil palm plants, Elaeis guineensis and Elaeis oleifera, produce palm oil and palm kernel oil and represent the highest yielding oil crop in the world. Palm oil has been forecasted to contribute to around a quarter of the world's demand for oil and fats and chemicals derived therefrom by the year 2020 (Rajanaidu and Jalani, World-wide performance of DXP planting materials and future prospects. In Proceedings of 1995 PORIM National Oil Palm Conference—Technologies in Plantation (1995), The Way Forward. Kuala Lumpur: Palm Oil Research Institute of Malaysia: 1-29). Due to the demand, there is a need to increase the quality and yield of palm oil and palm kernel oil and to rapidly develop new characteristics when required.

Oil palm has been identified as the most likely candidate for the development of a large scale renewable production plant for palm oil-derived chemicals (Ravigadevi et al. (2000) Genetic engineering in oil palm. In Advances in Oil Palm Research. (eds). Yusof, Jalani, and Chan Malaysia Palm Oil Board. 1:284-331). The ultimate aim is to genetically engineer the oil palm so as to modify its oil composition in order to expand its commercial and industrial applicability and meet production needs.

Conventional breeding approaches have been used for the introduction of new traits such as to generate dwarf palms and plants with high vitamin E and oleic acid content to meet industry and market requirements. However, the long generation time and the open pollinating behavior of oil palm contribute significantly to the slowness of the breeding process, which additionally also requires large amounts of plant material. Oil palm genetic engineering would enable the rapid introduction of new traits.

At present, Agrobacterium-mediated transformation (Izawati et al. (2009) J Oil Palm Res 21:643-652) and microprojectile bombardment (Parveez (2000) Production of transgenic oil palm (Elaesis guineensis Jacq) using biolistic techniques. In Molecular Biology of Wooded Plants (Eds. S. M. Jain and S. C. Minocha). Kluwer Academic Publishers 2:327-350) are being routinely used to introduce new traits into the oil palm. However, the traits obtained by both approaches can lack stability and reversion is a common occurrence. Furthermore, microprojectile bombardment is known to insert multiple copies of a transgene into the genome of transgenic plants.

There is a need to develop more efficacious methods for genetically manipulating plants of Elaeis sp.

### SUMMARY OF INVENTION

The present disclosure teaches a protocol for the regeneration of plants of the genus Elaeis from protoplasts derived from cells from embryogenic suspension cultures. In an embodiment, the protoplasts are genetically manipulated and then used to regenerate Elaeis plants with desired traits and beneficial properties.

Enabled herein is a method for regenerating a plant of the genus Elaeis from a protoplast, the method comprising isolating the protoplast from a cell of an embryogenic suspension culture and culturing the protoplast in a growth medium supplemented with selected plant growth regulators comprising auxins and cytokinins for a time and under conditions sufficient for a plant to form by somatic embryogenesis. In an embodiment, the growth medium is a Y3-based growth medium supplemented with a source of phosphorous and potassium such as but not limited to potassium dihydrogen phosphate or its equivalent. The selected plant growth regulators comprise the auxin indole-3-butyric acid (IBA) and the cytokinins gibberellic acid A3 (GA3) and 2-γ-dimethylallylaminopurine (2iP) or an equivalent of any one or more thereof at a concentration of from about 1 μM to about 20 μM. In an embodiment, naphthalene acetic acid, 6-benzylaminopurine, zeatin (Zea) and/or indole-3-acetic acid (IAA) is/are also included together with one or more of ascorbic acid (AA), silver nitrate and/or activated charcoal.

Taught herein is a method of regenerating a plant of the genus Elaeis from a protoplast, the method comprising isolating the protoplast from a cell from an embryogenic cell suspension culture using one or more enzymes which digest cell wall material, culturing the protoplast in a Y3-based medium comprising a source of phosphorous and potassium and up to about 2 μM of the plant growth regulators IBA, GA3 and 2iP for a time and under conditions sufficient for the protoplast to divide and develop a microcolony and then microcallus; culturing the microcallus in the presence of one or more of ascorbic acid, silver nitrate and/or activated charcoal and the plant growth regulators in order to produce embryogenic callus; and then transferring the embryogenic callus to a Y3-based liquid medium comprising about 1 μM NAA and 0.1 μM BA to promote somatic embryogenesis of embryos to form plantlets. In an embodiment, the source of phosphorous and potassium is potassium dihydrogen phosphate. In an embodiment, the protoplast is purified prior to regeneration into a plant.

The present specification further teaches, in an embodiment, embedding the protoplast in a solid phase in combination with the growth medium. Generally, the solid phase is a gelatinous polysaccharide such as but not limited to agarose or alginate.

Usefully, the protoplast is genetically modified by the introduction of a nucleic acid molecule such as a construct comprising the nucleic acid molecule operably linked to a promoter and capable of expression in the protoplast or its progeny. Conveniently, nucleic acid molecules are introduced to a suspension of protoplasts in the presence of polyethylene glycol (PEG), generally together with a salt such as but not limited to MgCl₂.

Conveniently, nucleic acid molecules are introduced to protoplasts embedded in a gelatinous polysaccharide such as but not limited to agarose or alginate, by microinjection. Once a plant has regenerated from the genetically manipulated protoplasts, the expressed nucleic acid molecule confers an advantageous trait in all or selected cells of the plant. This trait may be constitutively expressed or developmentally regulated. Hence, the trait results from expression of the genetic modification. The instant disclosure extends to parts of genetically modified plants which comprise cells which express the genetic modification. Plant parts include leaf, root, stem, seed and reproductive parts.

Plants of genus Elaeis include Elais guineensis, Elaeis oleifera (Elaeis melanococca) and Elaeis occidentalis. Conveniently, the plant is oil palm, Elaeis guineensis or Elaeis oleifera. Further taught herein is a genetically modified plant of the genus Elaeis when regenerated from a genetically manipulated protoplast according to the methods herein described and oil or fat products derived therefrom or a novel product arising from the genetic modification. Examples of products include plant metabolites.

Products of the genetically modified plants including palm oil and palm kernel oil as well as reproductive parts and tissue culture material are also encompassed herein as are kits for the isolation and manipulation of protoplasts.

### BRIEF DESCRIPTION OF THE FIGURES

Some figures contain color representations or entities. Color photographs are available from the Patentee upon request or from an appropriate Patent Office. A fee may be imposed if obtained from a Patent Office.

FIGS. 1A through S are photographic representations of protoplasts, in isolated form or embedded in agarose, microcolonies and microcalli of Elaeis guineensis. Refer to Examples for a description of each field.

FIGS. 2A through H are photographic representations of compact and friable embryogenic cells, somatic embryos, embryoids and plantlets of Elaeis guineensis. Refer to Examples for a description of each field.

FIGS. 3A through C are photographic representations of polyethylene glycol (PEG)-mediated transformed oil palm protoplasts from 7 and 14 day subcultures (A,B) of 3 month old suspension cultures and from 4 month old suspension cultures (C).

FIGS. 4A through D are photographic representations of PEG-mediated transformed oil palm protoplasts with (A) 10 mM; (B) 25 mM; (C) 50 mM; and (D) 100 mM MgCl₂.6H₂O.

FIGS. 5A through C are photographic representations of PEG-MgCl₂.6H₂O-mediated transformed oil palm protoplasts incubated with DNA for (A) 15 minutes or (B) 30 minutes or (C) in the presence of carrier DNA for 30 minutes.

FIGS. 6A through E are photographic representations of PEG-mediated transformed oil palm protoplasts using (A) 25 μg DNA or (B) 50 μg DNA; and (C) 25% w/v PEG; (D) 40% w/v PEG; and (E) 50% w/v PEG.

FIGS. 7A through C are photographic representations of PEG-mediated transformed oil palm protoplasts in 25% w/v PEG, 50 μg DNA with (A) 45° C., 5 minute heat shock; (B) 6 days; or (C) 9 days after transformation.

FIGS. 8A through T are photographic representations of protoplasts embedded in alginate, microinjection workstation and expression of DNA in oil palm protoplasts after microinjection.

FIGS. 9A through F are photographic representations of alginate layer-embedded protoplasts injected with 100 ng/μL, 500 ng/μL or 1000 ng/μL DNA.

FIGS. 10A through H are photographic representations of oil palm microcolonies formed from protoplasts following DNA microinjection.

### DETAILED DESCRIPTION OF THE PREFERRED EMBODIMENTS

Throughout this specification, unless the context requires otherwise, the word “comprise” or variations such as “comprises” or “comprising”, will be understood to imply the inclusion of a stated element or integer or method step or group of elements or integers or method steps but not the exclusion of any element or integer or method step or group of elements or integers or method steps.

As used in the subject specification, the singular forms “a”, “an” and “the” include singular and plural aspects unless the context clearly dictates otherwise. Thus, for example, reference to “a protoplast” includes a single protoplast, as well as two or more protoplasts; reference to “an Elaeis plant” includes a single plant, as well as two or more plants; reference to “the aspect” includes a single or multiple aspects taught by the disclosure. Aspects disclosed herein are encompassed by the term “invention”. All aspects of the invention are enabled within the width of the claims.

The present disclosure teaches the regeneration of a plant of the genus Elaeis from a protoplast. The ability to regenerate Elaeis plants from protoplasts enables genetic manipulation of the protoplasts in order to generate plants with desired traits.

Hence, an aspect taught herein is a method for regenerating a plant of the genus Elaeis, the method comprising isolating a protoplast from a cell from an embryogenic suspension culture and culturing the protoplast in a growth medium with selected plant growth regulators for a time and under conditions sufficient for a plant to regenerate by somatic embryogenesis. Generally, the growth medium comprises a source of phosphorous and potassium such as but not limited to potassium dihydrogen phosphate. In an embodiment, the growth medium comprises IBA, GA3 and 2iP. In an embodiment, the growth medium comprises NAA, BA, Zea and/or IAA as well as one or more of ascorbic acid (AA), silver nitrate and/or activated charcoal (AA).

Reference to “isolating” a protoplast includes “purifying” a protoplast or at least substantially purifying the protoplast. Reference to “a protoplast” includes a group or colony of protoplasts as well as a single protoplast. In an embodiment, the protoplasts are subject to genetic manipulation. The ability to genetically modify the protoplasts enables the development of plants or plant products with selected traits. Such traits include increasing yield of a plant product, producing a product not normally produced by the plant, modifying the composition of a plant product, and conferring disease resistance.

Hence, enabled herein is a method for generating a genetically modified plant of the genus Elaeis, the method comprising isolating a protoplast from a cell from an embryogenic suspension culture, introducing a nucleic acid molecule into the protoplast and culturing the protoplast in a growth medium with selected plant growth regulators for a time and under condition sufficient for a plant to form by somatic embryogenesis. In an embodiment, the growth medium comprises a source of phosphorous and potassium such as but not limited to potassium dihydrogen phosphate. In an embodiment, the growth medium comprises IBA, GA3 and 2iP. In an embodiment, the growth medium comprises NAA, BA, Zea and/or IAA as well as one or more of ascorbic acid (AA), silver nitrate and/or activate charcoal (AA).

In an embodiment, a nucleic acid molecule is introduced to a suspension of protoplasts in the presence of PEG, generally in the presence of a PEG-salt, such as but not limited to PEG-MgCl₂ (e.g. PEG- MgCl₂.6H₂O). In another embodiment, the nucleic acid molecule is introduced by microinjection of a protoplast embedded in a gelatinous polysaccharide such as agarose or alginate.

Hence, taught herein is a method for generating a genetically modified plant of the genus Elaeis, the method comprising generating a preparation of protoplasts and contacting the protoplasts with a sample of nucleic acid to be used to genetically modify the plant in the presence of polyethylene glycol (PEG) for a time and under conditions sufficient for the protoplast to be transformed by the nucleic acid and then regenerating a plant from the protoplast. In an embodiment, the PEG is a PEG-salt such as PEG-NgCl₂ (e.g. PEG-MgCl₂.6H₂O).

Further enabled herein is a method for generating a genetically modified plant of the genus Elaeis, the method comprising generating a preparation of protoplasts and subjecting individual protoplasts to microinjection with a sample of nucleic acid to be used to genetically modify the plant for a time and under conditions sufficient for the protoplast to be transformed by the nucleic acid and then regenerating the plant.

Progeny and later generations of the regenerated plants which express the trait introduced by the nucleic acid molecule are also contemplated herein as well as plant parts comprising cells which express the trait or genetic modification leading to the trait. A “plant part” includes a leaf, root, stem, seed and reproductive part.

In an embodiment, the protoplasts are from a 5-10 day subculture of 3 month embryogenic suspension culture. By “5-10” days means 5, 6, 7, 8, 9 or 10 days or a time period inbetween. Protoplasts from 7 day subculture of 3 month old suspension culture is generally useful. In an embodiment, for PEG-mediated transformation, PEG 4000 is used at a concentration of from 20-30% w/v which includes 20, 21, 22, 23, 24, 25, 26, 27, 28, 29 or 30%. An amount of 25% is useful. In an embodiment, 40-60 mM salt is included with the PEG, salt including MgCl₂.6H₂O. An amount of 50 mM MgCl₂.6H₂O is particularly useful. In an embodiment, the transformation process includes a heat shock step of 40-50° C. for 1-10 minutes including 40, 41, 42, 43, 44, 45, 46, 47, 48, 49 or 50° C. for 1, 2, 3, 4, 5, 6, 7, 8, 9 or 10 minutes followed by cooling down such as on ice. From about 15 to 100 μg of nucleic acid is used for the PEG-mediated transformation including 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 μg. Amounts of from 25-50 μg are useful.

For microinjection, conveniently, the gelatinous polysaccharide is agarose or alginate. An amount of 0.5-2.0% w/v alginate is particularly useful including 0.5, 0.6, 0.7, 0.8, 0.9, 1.0, 1.1, 1.2, 1.3, 1.4, 1.5, 1.6, 1.7, 1.8, 1.9 or 2.0% w/v. Useful nucleic acid concentrations include 0.5-2 ng/μL nucleic acid preparation which encompasses 0.5, 0.6, 0.7, 0.8, 0.9, 1.0, 1.1, 1.2, 1.3, 1.4, 1.5, 1.6, 1.7, 1.8, 1.9 or 2.0 ng/μL nucleic acid. An amount of 1 ng/μL is useful. Conveniently, microinjection is into the cytoplasm rather than the nucleus.

Generally one or a combination of enzymes is/are used to digest cell wall material. Such enzymes include a cellulase, pectinase, hydrolase and/or a glycosidase or their functional equivalents.

In an embodiment, the protoplast is cultured within a gelatinous polysaccharide such as an agarose or alginate solid phase. This includes being embedded within and in fluid contact with a medium. According to this embodiment, the purified protoplasts are resuspended in the growth medium with the selected plant growth regulators and from about 0.3% w/v to about 5% w/v polysaccharide. The percentage of polysaccharide varies depending on the number of protoplast, extent of manipulation performed on the protoplasts and the species of Elaeis but includes 0.4, 0.5, 0.6, 0.7, 0.8, 0.9, 1, 1.5, 2, 2.5, 3, 3.5, 4, 4.5 and 5% w/v. An amount of about 0.6±0.05% w/v agarose or alginate is useful according to an embodiment of the instant disclosure. Aliquots of the composition of protoplasts, growth medium, plant growth regulators and gelatinous polysaccharide are placed in a container for solidification or gelling. An osmoticum solution is in fluid contact with the solid phase composition which is replaced after the formation of microcolonies with a growth medium.

The growth medium is selected based on the protoplasts, their number, the manipulation performed and species of Elaeis. In an embodiment, the growth medium is a Y3-based medium comprising macroelements, microelements, carbohydrates, vitamins, amino acids and other organics. Y3 medium is described by Eewens (1976) Physiol Plant 36:23-238. A modified Y3 medium is described by Teixeira et al. (1995) Plant Cell, tissue and Organ Culture 40:405-411. In an embodiment, the macroelements are selected from NH₄NO₃, NH₄Cl, KNO₃, KCl, CaCl₂.2H₂O, MgSO₄.7H₂O, KH₂PO₄ and/or NaH₂PO₄.H₂O or varying other salts or equivalents thereof. The microelements include MnSO₄.4H₂O, ZnSO₄.7H₂O, H₃BO₃, Kl, CuSO₄.5H₂O, CoCl₂.6H₂O, Na₂MoO₄.2H₂O, NiCl₂.6H₂O and/or NaFeEDTA or other salts or equivalents thereof. Carbohydrates include sucrose, glucose, mannitol, sorbitol, fructose, mannose, maltose, dextrose and/or myo-inositol or an equivalent thereof.

In the practice of the instant method, the presence of potassium dihydrogen phosphate is highly desired as a source of phosphorous and potassium.

Vitamins include one or more of thiamine. HCl, pyridoxine HCl, nictoinic HCl, nicotinamide, calcium pantothenate, biotine, p-aminobenzoic acid, choline chyloride and/or ascorbic acid or an equivalent thereof. Amino acids include one or more of L-glutamine, L-asparagine, L-alanine, BSA, glycine, PVP-40 and/or L-cysteine or an equivalent thereof. Other organics include MES and/or PEG4000 or an equivalent thereof.

Taught herein is a Y3-based medium comprising 400-600 mg/l NH₄Cl, 1800-2200 mg/l, KNO₃, 1100-1600 mg/ml KCl, 250-350 mg/ml CaCl₂.2H₂O, 230-280 mg/ml MgSO₄.7H₂O, optionally 290-350 mg/ml NaH₂PO₄.H₂O, 8-15 mg/ml MnSO₄.4H₂O, 5-8 mg/ml ZnSO₄.7H₂O, 1-5 mg/ml H₃BO₃, 5-10 mg/ml Kl, 0.1 to 0.25 mg/ml CuSO₄.5H₂O, 0.1 to 0.5 mg/ml, CoCol₂.6H₂O, 0.1 to 0.5 mg/ml Na₂MoO₄.2H₂O, 0.001 to 0.005 mg/ml NiCl₂.6H₂O, 30-40 mg/ml NaFeEDTA, 10 to 100 g/l of sucrose, glucose and/or maltose, optimally 0.5 to 0.2 g/l myo-inositol, 0.1 to 15 mg/ml of three or more of thiamine HCl, pyridoxine HCl, nicotinic HCl, nicotinamide, calcium pantothenate, biotine, p-aminobenzoic acid, choline chloride and/or ascorbic acid, 80-250 mg/l of L-glutamine, L-asparagine, L-alginine, BSA, glycine and/or L-cysteine and optionally 4000-6000 mg/l PVP-40 and 200-400 mg/l of MES and/or PEG4000.

Taught herein are compositions of plant growth regulators comprising auxins naphthaleneacetic acid (NAA), 2,4-dichlorophenoxy acetic acid (2,4-D), indole-3-acetic acid (IAA) and indole-3-butyric acid (IBA) and cytokinins zeatin (Zea), gibberellic acid A3 (GA3), 6-benzylaminopurine (BA) and 2-γ-dimethylallylaminopurine (2iP) or an equivalent thereof.

The compositions of useful media of the Y3-type are listed in Tables 2 and 3.

The instant specification is instructional on a method of regenerating a plant of the genus Elaeis from a protoplast, the method comprising isolating the protoplast from a cell from an embryogenic cell suspension culture using one or more enzyme which digest cell wall material, culturing the protoplast in a Y3-based medium comprising a source of phosphorous and potassium and up to about 2 μM of the plant growth regulators IBA, GA3 and 2iP for a time and under conditions sufficient for the protoplast to divide and develop microcolonies and then microcallus; culturing the microcallus in the presence of one or more of ascorbic acid, silver nitrate and/or activated charcoal and the plant growth regulators in order to produce embryogenic callus; and then transferring the embryogenic callus to a Y3-based liquid medium comprising about 1 μM NAA and 0.1 μM BA to promote somatic embryogenesis of embryos to form plantlets.

Further taught herein is a method for regenerating a plant of the genus Elaeis, the method comprising isolating a cell from an embryogenic suspension culture, treating the cell with an enzyme preparation in order to digest cell wall material, isolating a protoplast from the cell and culturing the protoplast in the presence of a Y3-based medium supplemented with at least 1 μM of the plant growth regulators NAA, 2,4-D, IBA, GA3 and/or 2iP for a time and under conditions sufficient for microcallus to form from a microcolony and then permitting a plantlet to form on solid media. In an embodiment, from 2 to 20 μM of the plant growth regulators is provided. In an embodiment, the growth medium comprises IBA, GA3 and 2iP. In an embodiment, the growth medium comprises NAA, BA, Zea and/or IAA as well as one or more of ascorbic acid (AA), silver nitrate and/or activate charcoal (AA). In another embodiment, the concentration of plant growth regulators is as described in Table 3.

Further enabled herein is a method of regenerating a plant of the genus Elaeis from a genetically modified protoplast, the method comprising culturing the protoplast in a growth medium with selected plant growth regulators for a time and under conditions sufficient for a plantlet to form on solid media by somatic embryogenesis.

In an embodiment, the method comprises culturing the genetically modified protoplast in the presence of a Y3-based medium supplemented with at least 1 μM of the plant growth regulators NAA, 2,4-D, IBA, GA3 and/or 2iP for a time and under conditions sufficient for microcallus to form from a microcolony and then permitting plantlets to form a solid media.

In an embodiment, the method comprises a method of regenerating a genetically modified protoplast, the method comprising isolating a protoplast from a cell from an embryogenic cell suspension culture using one or more enzymes which digest cell wall material, introducing genetic material into the protoplast, culturing the protoplast in a Y3-based medium comprising potassium dihydrogen phosphate and up to about 2 μM of the plant growth regulators IBA, GA3 and 2iP for a time and under conditions sufficient for the protoplast to divide and develop microcolonies and then microcallus; culturing the microcallus in the presence of one or more of ascorbic acid, silver nitrate and/or activated charcoal and the plant growth regulators in order to produce embryogenic callus; and then transferring the embryogenic callus to a Y3-based liquid medium comprising about 1 μM NAA and 0.1 μM BA to promote somatic embryogenesis of embryos to form plantlets.

In accordance with these embodiments, the protoplasts may be additionally subjected to a purification step prior to culturing. In an embodiment, the protoplast is cultured in gelatinous solid phase culture such as agarose or alginate. For a genetically modified protoplast, the protoplast is either subject to nucleic acid transformation in the presence of PEG or the protoplasts are embedded in an alginate layer or an agarose layer and then subject to nucleic acid microinjection. The growth medium may or may not have an agent to provide selective pressure for a genetically modified protoplast.

Hence, this aspect of the disclosure encompasses isolated or substantially purified nucleic acid molecules for use in genetically modifying an Elaeis sp protoplast.

Another aspect enabled herein is a method for expressing a nucleic acid molecule in a plant and/or plant cell of the genus Elaeis, the method comprising introducing to a protoplast, a nucleic acid construct comprising a heterologous nucleotide sequence of interest operably linked to a promoter and regenerating a plant from the protoplast by the methods herein described.

As used herein, the term “construct” and/or “vector” and/or “plasmid” refers to a nucleic acid molecule capable of carrying another nucleic acid to which it has been linked or inserted. Particular vectors are those capable of expression of nucleic acids contained within. Vectors capable of directing the expression of genetic material to which they are operatively linked are referred to herein as “expression vectors.” In general, expression vectors of utility in recombinant nucleic acid techniques are often in the form of “plasmids” which refer generally to circular double stranded nucleic acid loops which, in their vector form, are not bound or inserted in the chromosome. In the present specification, “plasmid” and “vector” are used interchangeably. In particular, the plasmid or vector comprises a promoter and either a heterologous nucleotide sequence operably linked thereto or having restriction endonuclease means to insert a heterologous nucleotide sequence in operable linkage to the promoter. By “restriction endonuclease means” is meant one or more restriction endonuclease sites which can be used to linearize a covalently closed circular plasmid in order to re-ligate in the presence of a heterologous nucleotide sequence such that the heterologous nucleotide sequence is operably linked.

The term “genetic material” includes a “gene” which is used in its broadest sense and encompasses cDNA corresponding to the exons of a gene. Accordingly, reference herein to a “gene” is to be taken to include:
(i) a classical genomic gene consisting of transcriptional and/or translational regulatory sequences and/or a coding region and/or non-translated sequences (i.e. introns, 5′- and 3′-untranslated sequences);
(ii) mRNA or cDNA corresponding to the coding regions (i.e. exons) and 5′- and 3′-untranslated sequences of the gene; and/or
(iii) genetic material which when transcribed gives rise to mRNA or other RNA species including microRNA or after translation gives rise to a peptide, polypeptide or protein.

The terms “genetic material” and “gene” are also used to describe synthetic or fusion molecules encoding all or part of a functional product. The term “genetic material” also encompasses a gene or such molecules as RNAi, ssRNA, dsRNA, microRNA and the like.

The genetic material may be in the form of a genetic construct comprising a gene or nucleic acid molecule to be introduced into an Elaeis protoplast operably linked to a promoter and optionally operably linked to various regulatory sequences.

The genetic material for use herein may comprise a sequence of nucleotides or be complementary to a sequence of nucleotides which comprise one or more of the following: a promoter sequence, a 5′ non-coding region, a cis-regulatory region such as a functional binding site for transcriptional regulatory protein or translational regulatory protein, an upstream activator sequence, an enhancer element, a silencer element, a TATA box motif, a CCAAT box motif, or an upstream open reading frame, transcriptional start site, translational start site, and/or nucleotide sequence which encodes a leader sequence.

The term “5′ non-coding region” is used herein in its broadest context to include all nucleotide sequences which are derived from the upstream region of an expressible gene, other than those sequences which encode amino acid residues which comprise the polypeptide product of the gene, wherein the 5′ non-coding region confers or activates or otherwise facilitates, at least in part, expression of the gene.

Reference herein to a “promoter” is to be taken in its broadest context and includes the transcriptional regulatory sequences of a classical genomic gene, including the TATA box which is required for accurate transcription initiation, with or without a CCAAT box sequence and additional regulatory elements (i.e. upstream activating sequences, enhancers and silencers) which alter expression of genetic material in response to developmental and/or environmental stimuli, or in a tissue-specific or cell-type-specific manner. An Elaeis TCTP promoter is an example of a suitable promoter as is an 35S or other promoter such as a coconut foliar decay virus promoter. The promoter is usually, but not necessarily, positioned upstream or 5′, of genetic material, the expression of which it regulates. This is referred to as the promoter being operably linked to a particular nucleotide sequence.

In the present context, the term “promoter” is also used to describe a synthetic or fusion promoter molecule, or derivative thereof which confers, activates or enhances expression of genetic material.

The term “operably connected” or “operably linked” or “operatively linked” in the present context means placing a genetic material under the regulatory control of the promoter which then controls expression of this material. The promoter is generally positioned 5′ (upstream) to the genes which they control. In an embodiment, the function of the promoter is constitutive. Alternatively, the promoter is inducible and/or tissue specific.

The promoter sequence, when assembled within a DNA construct such that the promoter is operably linked to a nucleotide sequence of interest, enables expression of the nucleotide sequence in the protoplast stably transformed with this DNA construct as well as progeny or relatives of protoplast. As indicated above, the term “operably linked” is intended to mean that the transcription or translation of the heterologous nucleotide sequence is under the influence of the promoter sequence. “Operably linked” is also intended to mean the joining of two nucleotide sequences such that the coding sequence of each DNA fragment remains in the proper reading frame. In this manner, the nucleotide sequence for a promoter is provided in a DNA construct along with the nucleotide sequence of interest, typically a heterologous nucleotide sequence, for expression in the Elaeis plant of interest. The expression may be in any or all cells or in specific tissues. The term “heterologous nucleotide sequence” is intended to mean a sequence that is not naturally operably linked with the promoter sequence. While this nucleotide sequence is heterologous to the promoter sequence, it may be homologous or native or heterologous or foreign, to the Elaeis plant host.

Methods disclosed herein are useful for genetic engineering of Elaeis plants, e.g. for the production of a transformed or transgenic plant, to express a phenotype of interest. As used herein, the terms “transformed plant” and “genetically modified plant” refer to a Elaeis plant regenerated from a protoplast which comprises within its genome a heterologous polynucleotide. It includes an initially modified Elaeis plant as well as its progeny which carry the same genetic modification. Generally, the heterologous polynucleotide is stably integrated within the genome of a genetically modified or transformed Elaeis plant such that the polynucleotide is passed on to successive generations. The heterologous polynucleotide may be integrated into the genome or be part of a recombinant DNA construct. It is to be understood that as used herein the terms “genetically modified” and “transgenic” includes any protoplast, cell, cell line, callus, tissue, plant part, or plant the genotype of which has been altered by the presence of heterologous nucleic acid including those transgenics initially so altered as well as those created by sexual crosses or asexual propagation from the initial transgenic plant. “Genetically modified” and “transgenic” as used herein do not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation. A transgenic “event” is produced by transformation of protoplasts with a heterologous DNA construct, including a nucleic acid construct which comprises a transgene of interest, the regeneration of a population of a plant resulting from the insertion of the transgene into the genome of the plant, and selection of a particular plant characterized by expression of the introduced DNA. An event is characterized phenotypically by the expression of the transgene. At the genetic level, an event is part of the genetic makeup of a plant. The term “event” also refers to progeny produced by a sexual outcross between the transformant and another variety that include the heterologous DNA.

As used herein, the term “plant” includes reference to whole Elaeis plants, plant organs (e.g., leaves, stems, roots, etc.), seeds, plant cells, and progeny of same. Parts of transgenic Elaeis plants are to be understood within the scope of the embodiments to comprise, for example, plant, protoplasts, cells, tissues, callus, embryos as well as flowers, stems, fruits, ovules, leaves, or roots originating in transgenic plants or their progeny previously transformed with a DNA molecule of the embodiments, and, therefore, consisting at least in part of transgenic cells. As used herein, the term “plant cell” includes, without limitation, protoplasts, seeds suspension cultures, embryos, meristematic regions, callus tissue, leaves, roots, shoots, gametophytes, sporophytes, pollen, and microspores.

The ability to regenerate Elaeis plants from genetically modified protoplasts enables heterologous nucleotide sequences to be expressed in all or selected tissues of an Elaeis plant. Thus, the heterologous nucleotide sequence may be a structural gene encoding a protein of interest. Genes of interest are reflective of the commercial markets and interests of those involved in the development of palm oil or palm kernel oil plant crops. General categories of genes of interest for the embodiments include, for example, those genes involved in information, such as zinc fingers, those involved in communication, such as kinases, and those involved in housekeeping, such as heat shock proteins. More specific categories of transgenes, for example, include genes encoding proteins conferring resistance to abiotic stress, such as drought, temperature, salinity, and toxins such as pesticides and herbicides, or to biotic stress, such as attacks by fungi, viruses, bacteria, insects, and nematodes, and development of diseases associated with these organisms. The modification may also lead to increased carbon sink in reproductive tissue. Various changes in phenotype are of interest including modifying expression of a gene in a plant, altering a plant's pathogen or insect defense mechanism, increasing the plant's tolerance to herbicides, altering plant development to respond to environmental stress, and the like. The results can be achieved by providing expression of heterologous or increased expression of endogenous products in plants. Alternatively, the results can be achieved by providing for a reduction of expression of one or more endogenous products, particularly enzymes, transporters, or cofactors, or affecting nutrients uptake in the plant. These changes result in a change in phenotype of the transformed plant. It is recognized that any gene of interest can be operably linked to the promoter sequences of the embodiments and expressed in a plant.

In an embodiment, the genetically modified palm oil plant is modified to be a dwarf plant or produces oil with a high vitamin E or oleic acid content.

A DNA construct comprising a gene of interest can be used with transformation techniques, such as those described below, to create disease or insect resistance in susceptible plant phenotypes or to enhance disease or insect resistance in resistant plant phenotypes or to produce a modified oil to meet market needs. Accordingly, the embodiments encompass methods that are directed to protecting Elaeis plants against fungal pathogens, bacteria, viruses, nematodes, insects, and the like. By “disease resistance” is intended that the plants avoid the harmful symptoms that are the outcome of the Elaeis plant-pathogen interactions.

Disease resistance and insect resistance genes such as lysozymes, cecropins, maganins, or thionins for anti-bacterial protection, or the pathogenesis-related (PR) proteins such as glucanases and chitinases for anti-fungal protection, or Bacillus thuringiensis endotoxins, protease inhibitors, collagenases, lectins, and glycosidases for controlling nematodes or insects are all examples of useful gene products.

Genes encoding disease resistance traits include detoxification genes, such as against fumonisin (U.S. Pat. No. 5,792,931), avirulence (avr) and disease resistance (R) genes (Jones et al. (1994) Science 266:789; Martin et al. (1993) Science 262:1432; Mindrinos et al. (1994) Cell 78:1089); and the like.

The present disclosure may also be used to express genes in a root-preferred manner which may include, for example, insect resistance genes directed to those insects which primarily feed on the roots of Elaeis plants. Such insect resistance genes may encode resistance to pests that have great yield drag such as various species of rootworms, cutworms, and the like. Such genes include, for example, Bacillus thuringiensis toxic protein genes (U.S. Pat. Nos. 5,366,892; 5,747,450; 5,737,514; 5,723,756; 5,593,881; and Geiser et al. (1986) Gene 48:109); lectins (Van Damme et al. (1994) Plant Mol. Biol. 24:825); and the like. Herbicide resistance traits may be introduced into a plant by genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta, or other such genes known in the art. The bar gene encodes resistance to the herbicide basta, the nptII gene encodes resistance to the antibiotics kanamycin and geneticin, and the ALS gene encodes resistance to the herbicide chlorsulfuron.

Agronomically important traits which affect quality of palm oil products, such as levels and types of oils, saturated and unsaturated, quality and quantity of essential amino acids, levels of cellulose, starch, and protein content can be genetically altered using the methods herein described. Modifications include increasing content of oleic acid, saturated and unsaturated oils, increasing levels of lysine and sulfur, providing essential amino acids, and modifying starch.

Exogenous products include plant enzymes and products as well as those from other sources including prokaryotes and other eukaryotes. Such products include enzymes, cofactors, hormones, and the like.

Examples of other applicable genes and their associated phenotype include the gene that encodes viral coat protein and/or RNA, or other viral or plant genes that confer viral resistance; genes that confer fungal resistance; genes that confer insect resistance; genes that promote yield improvement; and genes that provide for resistance to stress, such as dehydration resulting from heat and salinity, toxic metal or trace elements, or the like.

The heterologous nucleotide sequence operably linked to a promoter may also be an antisense sequence for a targeted Elaeis gene. The terminology “antisense DNA nucleotide sequence” is intended to mean a sequence that is in inverse orientation to the 5′-to-3′ normal orientation of that nucleotide sequence. When delivered into a plant cell, expression of the antisense DNA sequence prevents normal expression of the DNA nucleotide sequence for the targeted gene. The antisense nucleotide sequence encodes an RNA transcript that is complementary to and capable of hybridizing to the endogenous messenger RNA (mRNA) produced by transcription of the DNA nucleotide sequence for the targeted gene. In this case, production of the native protein encoded by the targeted gene is inhibited to achieve a desired phenotypic response. Modifications of the antisense sequences may be made as long as the sequences hybridize to and interfere with expression of the corresponding mRNA. In this manner, antisense constructions having 70%, 80%, 85% sequence identity to the corresponding antisense sequences may be used. Furthermore, portions of the antisense nucleotides may be used to disrupt the expression of the target gene. Generally, sequences of at least 10 nucleotides, 15 nucleotides, 20 nucleotides, 50 nucleotides, 100 nucleotides, 200 nucleotides, or greater may be used. Thus, the promoter sequences disclosed herein may be operably linked to antisense DNA sequences to reduce or inhibit expression of a native protein in the plant. “RNAi” refers to a series of related techniques to reduce the expression of genes (See for example U.S. Pat. No. 6,506,559). Older techniques referred to by other names are now thought to rely on the same mechanism, but are given different names in the literature. These include “antisense inhibition,” the production of antisense RNA transcripts capable of suppressing the expression of the target protein, and “co-suppression” or “sense-suppression,” which refer to the production of sense RNA transcripts capable of suppressing the expression of identical or substantially similar foreign or endogenous genes (U.S. Pat. No. 5,231,020). Such techniques rely on the use of constructs resulting in the accumulation of double stranded RNA with one strand complementary to the target gene to be silenced._-A promoter sequence may be used to drive expression of constructs that will result in RNA interference including microRNAs and siRNAs.

In preparing the DNA construct, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites. Restriction sites may be added or removed, superfluous DNA may be removed, or other modifications of the like may be made to the sequences of the embodiments. For this purpose, in vitro mutagenesis, primer repair, restriction, annealing, re-substitutions, for example, transitions and transversions, may be involved. Reporter genes or selectable marker genes may be included in the DNA constructs. Examples of suitable reporter genes known in the art can be found in, for example, Jefferson et al. (1991) In Plant Molecular Biology Manual, ed. Gelvin et al. (Kluwer Academic Publishers):1-33; DeWet et al. (1987) Mol. Cell. Biol. 7:725-737; Goff et al. (1990) EMBO J. 0:2517-2522; Kain et al. (1995) BioTechniques 79:650-655; and Chiu et al. (1996) Current Biology 6:325-330.

Selectable marker genes for selection of transformed cells or tissues can include genes that confer antibiotic resistance or resistance to herbicides. Examples of suitable selectable marker genes include, but are not limited to, genes encoding resistance to chloramphenicol (Estrella et al. (1983) EMBO J. 2:987-992); methotrexate (Estrella et al. (1983) Nature 303:209-213; Meijer et al. (1991) Plant Mol. Biol. 16:807-820); hygromycin (Waldron et al. (1985) Plant Mol. Biol. 5:103-108; Zhijian et al. (1995) Plant Science 708:219-227); streptomycin (Jones et al. (1987) Mol. Gen. Genet. 270:86-91); spectinomycin (Bretagne-Sagnard et al. (1996) Transgenic Res. 5:131-137); bleomycin (Hille et al. (1990) Plant Mol. Biol. 7:171-176); sulfonamide (Guerineau et al. (1990) Plant Mol. Biol. 75:127-136); bromoxynil (Stalker et al. (1988) Science 242:419-423); glyphosate (Shaw et al. (1986) Science 233:478-481); phosphinothricin (DeBlock et al. (1987) EMBO J. 6:2513-2518).

Other genes that could serve utility in the recovery of transgenic events but might not be required in the final product would include, but are not limited to, examples such as GUS (β-glucuronidase; Jefferson (1987) Plant Mol. Biol. Rep. 5:387 (green fluorescent protein [GFP]; Chalfie et al. (1994) Science 263:802), luciferase (Riggs et al. (1987) Nucleic Acids Res. 75(19):8115 and Luehrsen et al. (1992) Methods Enzymol. 276:397-414), and the maize genes encoding for anthocyanin production (Ludwig et al. (1990) Science 247:449).

The nucleic acid molecules of the embodiments are useful in methods directed to expressing a nucleotide sequence in an Elaeis plant. This may be accomplished by transforming including microinjecting an Elaeis protoplast with a DNA construct or transforming the protoplast in the presence of PEG, generally a PEG-salt solution (e.g. PEG-MgCl₂.6H₂O) and regenerating a stably transformed plant from the protoplast. The methods of the embodiments are also directed to inducibly expressing a nucleotide sequence in a plant. Those methods comprise transforming including injecting a protoplast with a DNA construct regenerating a transformed plant from the protoplast, and, if necessary, subjecting the plant to the required stimulus to induce expression. The DNA construct, can be used to transform any species of Elaeis. In this manner, genetically modified, i.e. transgenic or transformed, plants, plant protoplasts, plant cells, plant tissue, seed, root, and the like can be obtained.

As used herein, “construct” refers to a DNA molecule such as a plasmid, cosmid, or bacterial phage for introducing a nucleic acid molecule, for example, in an expression cassette, into a host protoplast. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance, or ampicillin resistance.

The methods described herein involve introducing a nucleic acid construct into Elaeis protoplast. As used herein “introducing” is intended to mean presenting to the plant the nucleotide construct in such a manner that the construct gains access to the interior of the cell. The methods herein do not depend on a particular method for introducing a nucleotide construct to an Elaeis protoplast, only that the nucleic acid construct gains access to the interior of at least one protoplast. Methods for introducing nucleic acid constructs into plants are known in the art including, but not limited to, stable transformation methods, transient transformation methods, microinjection, and virus-mediated methods. A “stable transformation” is one in which the nucleotide construct introduced into a plant integrates into the genome of the plant and is capable of being inherited by progeny thereof. “Transient transformation” means that a nucleotide construct introduced into a plant does not integrate into the genome of the plant. The nucleotide constructs of the embodiments may be introduced into plants by contacting plants with a virus or viral nucleic acids. Generally, such methods involve incorporating a nucleotide construct within a viral DNA or RNA molecule. Methods for introducing nucleotide constructs into plants and expressing a protein encoded therein, involving viral DNA or RNA molecules, are known in the art. See, for example, U.S. Pat. Nos. 5,889,191, 5,889,190, 5,866,785, 5,589,367, and 5,316,931). Two particularly useful protocols involve PEG-salt and microinjection.

Transformation protocols as well as protocols for introducing nucleotide sequences into plants may vary depending on the size of the nucleic acid molecule and the number of protoplasts available. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606), Agrobacterium-mediated transformation (U.S. Pat. Nos. 5,981,840 and 5,563,055), direct gene transfer (Paszkowski et al. (1984) EMBO J. 3:2717-2722), and ballistic particle acceleration (see, for example, U.S. Pat. Nos. 4,945,050; 5,879,918; 5,886,244; 5,932,782; Tomes et al. (1995) In Plant Cell, Tissue, and Organ Culture: Fundamental Methods, ed. Gamborg and Phillips (Springer-Verlag, Berlin); and McCabe et al. (1988) Biotechnology 6:923-926).

The present disclosure further teaches kits for facilitating the isolation of protoplasts from Elaeis sp and their regeneration into plants. The kits are generally in compartmental form comprising one or more compartments which contain medium or a reconstitutable form thereof for use in maintaining a suspension culture; a growth medium or a reconstitutable form thereof; agarose or alginate or other suitable gelatinous polysaccharide material to embed the protoplasts; cell culture containers; genetic molecules; and reagents.

Reference herein to Elaeis sp include Elaeis guineensis, Elaeis oleifera (Elaeis melanococca) and Elaeis occidentalis.

The instant disclosure further enables the use of a protoplast from a cell from an embryogenic suspension culture in the regeneration of a plant Elaeis sp. The use further comprises a growth medium comprising a source of phosphorous and potassium such as but not limited to potassium dihydrogen phosphate. The use further comprises a growth medium comprising IBA, GA3 and 2iP. The use further comprises a growth medium comprising one or more of NAA, BA, Zea and/or IAA. The instant disclosure further provides a business model comprising Elaeis tissue culture or reproductive material generated from genetically modified protoplasts and stored for sale to Elaeis breeders for use in generating Elaeis crops for beneficial properties.

Abbreviations used herein are defined in Table 1.

TABLE 1
Abbreviations

| Abbreviation | Definition |
|---|---|
| 2,4-D | 2,4-Dichlorophenoxyacetic acid |
| 2iP | 2-γ-Dimethylallylaminopurine |
| 5-CFDA, AM | 5-carboxyfluorescein diacetate, acetoxymethyl ester |
| AA | Ascorbic acid |
| AC | Activated charcoal |
| AgNO₃ | Silver nitrate |
| BA | 6-Benzylaminopurine |
| CE | Compact embryogenic callus |
| CFDV | Coconut foliar decay virus |
| CLSM | Confocal laser scanning microscopy |
| FE | Friable embryogenic callus |
| Fwt | Fresh weight |
| GA3 | Gibberellic acid A3 |
| hrGFP | Humanized renilla green fluorescent protein |
| IAA | Indole-3-acetic acid |
| IBA | Indole-3-butyric acid |
| KCl | Potassium chloride |
| KH₂PO₄ | Potassium dihydrogen phosphate |
| KNO₃ | Potassium nitrate |
| MgCl₂ | Magnesium chloride |
| NAA | Naphthaleneacetic acid |
| NH₄ | Ammonium |
| NH₄Cl | Ammonium chloride |
| NH₄NO₃ | Ammonium nitrate |
| NiCl₂•6H₂O | Nickel chloride |
| NO₃ | Nitrate |
| PEG | Polyethlyene glycol |
| PGRs | Plant growth regulators |
| Y35N5D2iP | Y3 medium supplemented with 5 μM NAA, |
| | 5 μM 2,4-D and 2 μM 2iP |
| Y31N0.1BA | Y3 medium supplemented with 1 μM NAA |
| | and 0.1 μM BA |

### EXAMPLES

Aspects disclosed herein are now further described by the following non-limiting Examples. In the Examples, the following Materials and Methods are employed.

Oil Palm Suspension Culture

Oil palm embryogenic cell suspensions were cultured in an 100 ml Erlenmeyer flask containing 50 ml Y3 (Eewens (1976) supra) liquid media (Table 2) supplemented with 5 μM 1-naphthaleneacetic acid (NAA), 5 μM 2,4-dichlorophenoxyacetic acid (2,4-D) and 2 μM 2-γ-dimethylallylaminopurine (2iP). This medium is designated “Y35N5D2iP”. The suspension cultures were incubated in the dark at 28° C. on a rotary shaker and agitated at 120 rpm. Half of the Y35N5D2iP liquid media in the flask cultures was discarded and replaced with fresh media about every 14 days.

Protoplast Isolation

Protoplasts were isolated from embryogenic cell suspension up to about 14 days after fresh media was added. The embryogenic cell suspension was collected by filtration with 300 μm nylon mesh and 0.5 g of embryogenic cells transferred into a 50 ml centrifuge tube containing 15 ml of filter-sterilized enzyme solution consisting of 2% v/v Celluclast (Sigma), 1% v/v Pextinex 3XL (Sigma), 0.5% w/v Cellulase onuzuka RIO (Duchefa), 0.1% w/v Pectolyase Y23 (Duchefa), 3% w/v KCl, 0.5% w/v CaCl₂.2H₂O and 3.6% w/v mannitol at pH 5.6. The embryogenic cells were resuspended in enzyme solution by inverting the centrifuge tube for 6-10 times. The centrifuge tube was placed in a horizontal condition and incubated in the dark without shaking at 26° C. for about 14 hours.

TABLE 2
Composition of the media used

| Media | | | | | | | | |
|---|---|---|---|---|---|---|---|---|
| component | ½ECI | ECI | ECII | ECIII | ECIV | ECV | ECVI | Y3 |
| Macroelement | | | | | | | | |
| (mg/L) | | | | | | | | |
| NH₄NO₃ | 825 | 1650 | 600 | 1650 | 1650 | 1650 | | |
| NH₄Cl | | | | | | | | 535 |
| KNO₃ | 950 | 1900 | 1900 | 1900 | 1900 | 1900 | 1900 | 2020 |
| KCl | | | 300 | | | | | 1492 |
| CaCl₂•2H₂O | 220 | 440 | 453 | 440 | 440 | 440 | 440 | 294 |
| MgSO₄•7H₂O | 185 | 370 | 146 | 370 | 370 | 370 | 370 | 247 |
| KH₂PO₄ | 85 | 170 | 170 | 420 | 170 | 170 | 170 | |
| NaH₂PO₄•H₂O | | | | | | | | 312 |
| Microelements | | | | | | | | |
| (mg/L) | | | | | | | | |
| MnSO₄•4H₂O | 11.15 | 22.3 | 10 | 22.3 | 22.3 | 22.3 | 22.3 | 11.2 |
| ZnSO₄•7H₂O | 4.3 | 8.6 | 2 | 8.6 | 8.6 | 8.6 | 8.6 | 7.2 |
| H₃BO₃ | 3.1 | 6.2 | 3 | 6.2 | 6.2 | 6.2 | 6.2 | 3.1 |
| Kl | 0.42 | 0.83 | 0.75 | 0.83 | 0.83 | 0.83 | 0.83 | 8.3 |
| CuSO₄•5H₂O | 0.013 | 0.026 | 0.026 | 0.026 | 0.026 | 0.026 | 0.026 | 0.16 |
| CoCl₂•6H₂O | 0.013 | 0.026 | 0.026 | 0.026 | 0.026 | 0.026 | 0.026 | 0.24 |
| Na₂MoO₄•2H₂O | 0.125 | 0.25 | 0.25 | 0.25 | 0.25 | 0.25 | 0.25 | 0.24 |
| NiCl₂•6H₂O | | | | | | | | 0.0024 |
| NaFeEDTA | 18.75 | 37.5 | 36.7 | 37.5 | 37.5 | 37.5 | 37.5 | 37.5 |
| Carbohydrates | | | | | | | | |
| (g/L) | | | | | | | | |
| Sucrose | 30 | 30 | 30 | 40 | | 5 | 30 | 45 |
| Glucose | | | | 7.2 | | 5 | | |
| Mannitol | | | | | | 5 | | |
| Sorbitol | | | | | | 5 | | |
| Fructose | | | | | | 5 | | |
| Mannose | | | | | | 5 | | |
| Maltose | | | | | | 5 | | |
| Dextrose | | | | | 30 | 5 | | |
| Myo-inositol | 0.1 | 0.1 | 0.1 | 0.1 | 0.1 | 0.1 | 0.1 | 0.1 |
| Vitamins (mg/L) | | | | | | | | |
| Thiamine HCL | 1 | 1 | 1 | 1 | 1 | 1 | 1 | 1 |
| Pyridoxine HCL | 1 | 1 | 1 | 1 | 1 | 1 | 1 | 1 |
| Nicotinic HCL | 1 | 1 | 1 | 1 | 1 | 1 | 1 | 1 |
| Nicotinamide | | | | | | | | |
| Ca- | | | | | | | | |
| Pantothenate | | | | | | | | |
| Biotine | | | | | | | | |
| p-Aminobenzoic | | | | | | | | |
| Choline chloride | | | | | | | | |
| Ascorbic acid | | | | | | | | |
| Amino acids | | | | | | | | |
| (mg/L) | | | | | | | | |
| L-Glutamine | 50 | 100 | 100 | 100 | 100 | 100 | 100 | 100 |
| L-Asparagine | 50 | 100 | 100 | 100 | 100 | 100 | 100 | 100 |
| L-Alginine | 50 | 100 | 100 | 100 | 100 | 100 | 100 | 100 |
| BSA | | | | 260 | | | | |
| Glycine | | | | | | | | 4 |
| PVP-40 | | | | | | | | |
| L-Cysteine | | | | | | | | |
| Other organics | | | | | | | | |
| (mg/L) | | | | | | | | |
| MES | 250 | 250 | 250 | 250 | 250 | 250 | 250 | 250 |
| PEG4000 | | | | | | | | |

| Media | | | | | | |
|---|---|---|---|---|---|---|
| component | Y3A | Y3B | Y3C | Y3D | Y3E | Y3F |
| Macroelement | | | | | | |
| (mg/L) | | | | | | |
| NH₄NO₃ | | | | | | |
| NH₄Cl | 535 | 535 | 535 | 535 | 535 | 535 |
| KNO₃ | 2020 | 2020 | 2020 | 2020 | 2020 | 2020 |
| KCl | 1492 | 1492 | 1492 | 1492 | 1492 | 1492 |
| CaCl₂•2H₂O | 294 | 294 | 294 | 294 | 294 | 294 |
| MgSO₄•7H₂O | 247 | 247 | 247 | 247 | 247 | 247 |
| KH₂PO₄ | 250 | | | 250 | 250 | 250 |
| NaH₂PO₄•H₂O | 312 | 312 | 312 | 312 | 312 | 312 |
| Microelements | | | | | | |
| (mg/L) | | | | | | |
| MnSO₄•4H₂O | 11.2 | 11.2 | 11.2 | 11.2 | 11.2 | 11.2 |
| ZnSO₄•7H₂O | 7.2 | 7.2 | 7.2 | 7.2 | 7.2 | 7.2 |
| H₃BO₃ | 3.1 | 3.1 | 3.1 | 3.1 | 3.1 | 3.1 |
| Kl | 8.3 | 8.3 | 8.3 | 8.3 | 8.3 | 8.3 |
| CuSO₄•5H₂O | 0.16 | 0.16 | 0.16 | 0.16 | 0.16 | 0.16 |
| CoCl₂•6H₂O | 0.24 | 0.24 | 0.24 | 0.24 | 0.24 | 0.24 |
| Na₂MoO₄•2H₂O | 0.24 | 0.24 | 0.24 | 0.24 | 0.24 | 0.24 |
| NiCl₂•6H₂O | 0.0024 | 0.0024 | 0.0024 | 0.0024 | 0.0024 | 0.0024 |
| NaFeEDTA | 37.5 | 37.5 | 37.5 | 37.5 | 37.5 | 37.5 |
| Carbohydrates | | | | | | |
| (g/L) | | | | | | |
| Sucrose | 40 | 40 | 40 | 30 | 40 | 29 |
| Glucose | 72 | | | 15 | 72 | 25.2 |
| Mannitol | | | | | | |
| Sorbitol | | | | | | |
| Fructose | | | | | | |
| Mannose | | | | | | |
| Maltose | | | | | | 25 |
| Dextrose | | | | | | |
| Myo-inositol | 0.1 | 0.1 | 0.1 | 0.1 | | 0.2 |
| Vitamins (mg/L) | | | | | | |
| Thiamine HCL | 1 | 2.5 | 1 | 1 | 1 | 1 |
| Pyridoxine HCL | 1 | 0.4 | 1 | 1 | 1 | 1 |
| Nicotinic HCL | 1 | 10 | 1 | 1 | 1 | 1 |
| Nicotinamide | | | | | 1 | 1 |
| Ca- | | | | | 1 | 1 |
| Pantothenate | | | | | | |
| Biotine | | | | | 0.1 | 0.1 |
| p-Aminobenzoic | | | | | | 0.5 |
| Choline chloride | | | | | | 0.5 |
| Ascorbic acid | | 250 | | | | |
| Amino acids | | | | | | |
| (mg/L) | | | | | | |
| L-Glutamine | 100 | 100 | 200 | 200 | 100 | 100 |
| L-Asparagine | 100 | | 100 | | | |
| L-Alginine | 100 | | 100 | | | |
| BSA | | | | | | |
| Glycine | 4 | | 4 | | | |
| PVP-40 | | 5000 | | | | |
| L-Cysteine | | 500 | | | | |
| Other organics | | | | | | |
| (mg/L) | | | | | | |
| MES | 250 | 250 | 250 | 250 | 250 | 250 |
| PEG4000 | | | | | 250 | |

Protoplast Purification

After incubation, the mixture was diluted with 15 ml of filter-sterilized washing solution consisting of 3% w/v KCl, 0.5% w/v CaCl₂.2H₂O and 3.6% w/v mannitol at pH 5.6. The diluted mixture was resuspended by inverting the centrifuge tube 3-5 times and then was filtered through a sterilized double layer miracloth (22 μM) by collection in a 50 ml centrifuge tube. The filtration step was repeated 2-3 times until all the undigested tissues, cell clumps and cell wall debris were removed. The centrifuge tube was centrifuged at 60×g for 5 minutes at 22° C. and the supernatant was removed. The protoplast pellet was resuspended by inverting the tube with addition of 10 ml washing solution, and then was centrifuged. After repeating 3 times with the washing step, the supernatant was removed completely, and the protoplast pellet was resuspended with 5 ml filter-sterilized Rinse solution consisting of 3% w/v KCl and 3.6% w/v mannitol at pH 5.6.

Protoplast Yield and Viability

The yield and viability of the purified protoplast were calculated with a Nageotte hematocytometer in 3 replicates for each independent experiment. The following formula was used as X=Y×10⁵/Z [X is the number of protoplasts per mL, Y is the average quantity of protoplasts in 5×1 mm², Z is the fresh weight (fwt) of plant material in grams] as the protoplast yield, whereas the viability was calculated as the number of protoplast fluorescing green after stained by 5-carboxyfluorescein diacetate, acetoxymethyl ester (5-CFDA, AM; Invitrogen) divided by the protoplast yield in percent. Cell wall formation was evaluated after stained by fluorescent brightener 28 (Sigma).

Protoplast Culture

Media Optimization

The purified protoplasts were cultured using different media (Table 2) either in liquid or embedded in agarose or alginate solidified media. The protoplasts were cultured at the density 1×10⁵ protoplasts/ml of media. Five ml rinse solution containing the purified protoplasts was allowed to settle for 20 minutes at room temperature. For liquid culture, the rinse solution was replaced with liquid media and 2 ml each dispensed into 24 wells culture plate. For agarose or alginate embedded solidified cultures, the protoplast pellet was resuspended with a double concentration of liquid media at the density 2×10⁵ protoplasts/ml. Agarose sea plaque (Duchefa) was dissolved at the concentration of 1.2% w/v by heating in distilled water containing of 0.1% w/v 2-N-morpholino ethanesulfonic acid (MES), and then the pH was adjusted to 5.7. The agarose solution was filter-sterilized and kept at 37° C. Equal volumes of suspension protoplasts and agarose were mixed by adjusting the final concentration to 0.6% w/v of agarose, and then 2 ml each of the mixtures was dispensed into 24 wells culture plate. The culture plate was placed at room temperature for an hour for agarose solidification. The protoplasts embedded in agarose solidified media in each well were covered with 500 μl of the same liquid media was used for preparation of agarose solidified cultures. The culture plates containing liquid or agarose solidified cultures were sealed and incubated at 28° C. in the dark. The culture was monitored microscopically everyday to observe the first and second cell division, and seven days intervals for microcolonies and microcalli formations. When alginate was used, the same method steps were employed.

Plant Growth Regulators (PGRs) Optimization

The PGRs optimization was performed on agarose solidified cultures. All PGRs used were prepared at the concentration of 1 μM/μl and the pH was adjusted to 5.7. The filter-sterilized PGRs were added in 24 wells culture plate at different combinations and concentration as indicated in Table 3.

TABLE 3
Composition of PGRs used

| PGR | | | | | | | | |
|---|---|---|---|---|---|---|---|---|
| combination | Auxin (μM) | | | | Cytokinin (μM) | | | |
| No. | NAA | 2,4-D | IAA | IBA | Zea | GA3 | B | 2iP |
| 1 | 1 | 11 | 1 | 11 | 1 | 11 | 1 | 11 |
| 2 | 2 | 10 | 2 | 10 | 2 | 10 | 2 | 10 |
| 3 | 3 | 9 | 3 | 9 | 3 | 9 | 3 | 9 |
| 4 | 4 | 8 | 4 | 8 | 4 | 8 | 4 | 8 |
| 5 | 5 | 7 | 5 | 7 | 5 | 7 | 5 | 7 |
| 6 | 6 | 6 | 6 | 6 | 6 | 6 | 6 | 6 |
| 7 | 7 | 5 | 7 | 5 | 7 | 5 | 7 | 5 |
| 8 | 8 | 4 | 8 | 4 | 8 | 4 | 8 | 4 |
| 9 | 9 | 3 | 9 | 3 | 9 | 3 | 9 | 3 |
| 10 | 10 | 2 | 10 | 2 | 10 | 2 | 10 | 2 |
| 11 | 11 | 1 | 11 | 1 | 11 | 1 | 11 | 1 |
| 12 | 10 | | 10 | 2 | 10 | 2 | 10 | 2 |
| 13 | 10 | 2 | | 2 | 10 | 2 | 10 | 2 |
| 14 | 10 | 2 | 10 | | 10 | 2 | 10 | 2 |
| 15 | 10 | 2 | 10 | 2 | | 2 | 10 | 2 |
| 16 | 10 | 2 | 10 | 2 | 10 | | 10 | 2 |
| 17 | 10 | 2 | 10 | 2 | 10 | 2 | | 2 |
| 18 | 10 | 2 | 10 | 2 | 10 | 2 | 10 | |
| 19 | 10 | 2 | | 2 | | 2 | | 2 |

Agarose Bead Cultures

The mixture of protoplasts and agarose was prepared by using the same procedure for preparation of agarose solidified cultures with the exception that the protoplast pellet was resuspended in Y3A liquid media supplemented with the optimum PGRs and 0.6% w/v agarose sea plaque. Agarose beads were prepared by dropping 200 μl of the mixture into a 60 mm×15 mm petri dish. After agarose solidification, 10 ml of 21% v/v osmoticum solution was added to Petri dish and incubated at 28° C. in the dark for 3-5 days. Three types of osmoticum solutions were used: sucrose, glucose and mannitol. Each was dissolved in water, adjusting the pH to 5.7 and then filter-sterilized. The osmoticum solution was replaced with different liquid media in shaking condition at 50 rpm by refreshing the media at 14-day intervals. When the formation of microcolonies was observed, Y3A liquid media was changed to Y3 liquid media and the agarose beads were cultured until the microcalli were detected. After the microcalli appeared visible to the naked eye, the agarose beads was cultured in Y35N5D2iP liquid media supplemented with different concentrations of ascorbic acid (AA: 50 mg/l, 100 mg/l, 150 mg/l, 200 mg/l and 400 mg/l), silver nitrate (AgNO3: 5 mg/l, 10 mg/l, 15 mg/l) and activated charcoal (AC: 0.1 g/l, 0.3 g/l, 0.5 g/l and 1.0 g/l). The cultures were continued until the microcalli growth to embryogenic calli.

Division, Microcolonies and Microcalli Frequencies

Protoplast division frequency was calculated by counting the number of protoplasts divided by the total number of protoplasts in one representative microscope field. Three microscopic fields were averaged to represent one experiment in which the experiment was performed in 3 replicates to give an average range of protoplast division frequency. A similar calculation procedure was used for microcolonies and microcalli formation frequencies.

Plant Regeneration of Protoplasts-Derived Embryogenic Calli

The agarose beads were transferred to a Y3 solid media (0.6% w/v plant agar) when microcalli developed to 5-10 mm in size of whitish and yellowish embryogenic calli. The agarose beads were maintained on Y3 solid media supplemented with different concentrations of a combination of NAA (0.5-10 μM) and 6-benzylaminopurine (BA) [0.1-5 μM] until the formation of embryos was observed. The agarose beads containing the embryogenic calli were incubated at 28° C. in the dark and were subcultured every 30 days in fresh medium. The embryos were transferred onto ECI solid media supplemented with the optimum PGRs of NAA and BA, and then were incubated at 28° C. in the light until small plantlets were produced. Small plantlets were transferred onto ECI solid media supplemented with 0.1 μM NAA for root formation and for development into plants.

Polyethylene Glycol (PEG) Mediated Transformation

Protoplasts were isolated from a 3 month old embryogenic suspension culture at 7 and 14 days after subculture following the protocol described above. After twice washing with Washing solution, the supernatant was mostly removed leaving about 1 ml Washing solution and incubated at room temperature for 10 minutes. The protoplast suspension was then incubated at 45° C. for 5 minutes and immediately placed on ice for 1 minute, then incubated at room temperature for 10 minutes. A 500 μl aliquot of the protoplast suspension was then placed as a single droplet in the middle of a 60 mm×15 mm petri dish (no. 628102, Greiner Bio-One, Germany). The protoplast drop was surrounding by 5 drops of 100 μL PEG-MgCl solution containing 25% w/v PEG 4000, 50 μM MgCl₂.6H₂O which were dissolved in Rinse solution adjusted to pH 6.0. Fifty μg of CFDV-hrGFP (humanized renilla green fluorescent protein gene driven by coconut foliar decay virus promoter) plasmid DNA was added slowly to the protoplasts drop, mixed by stirring with 200 μL tip and incubated at room temperature in the dark. After incubation for 10 minutes, the DNA-protoplast drop was sequentially mixed with each of PEG-MgCl drop by stirring with 200 μL tip and incubated for another 30 minutes, then 4 mL Washing solution was added drop by drop and incubated in the dark at 26° C. for 9 days.

Confocal Laser Scanning Microscopy (CLSM)

Protoplasts were observed using a CLSM (Leica TCS 5 SP5 X) and visualized by Leica Microsystem LAS AF. GFP and autofluorescence of the chlorophyll were excitated at 488 nm and 543 nm wavelengths, respectively. The emission filters were 500-600 nm and 675-741 nm for chlorophyll autofluorescence. PEG-mediated transfection efficiency was calculated as the percentage of the number of protoplast fluorescing green (GFP positive protoplasts) divided by the total number of protoplasts in one representative microscope field. The calculation was performed three times for a total of not less than 200 protoplasts.

DNA Microinjection Mediated Transformation

Protoplast Isolation

Protoplasts were isolated from a 7 day subculture of a 3 month old embryogenic suspension culture as described above. After twice repeating the washing step, the supernatant was mostly removed leaving 3 ml Washing solution. The centrifuge tube containing the protoplast suspension was incubated in a vertical position in the dark for 24 hours at 28° C. After incubation, the protoplast suspension was diluted by 10 ml of Rinse solution and resuspended by inverting the centrifuge tube for 3-5 times, and then centrifuged at 60×g for 5 minutes at 22° C. After repeating the rinsing step, the supernatant was removed completely and the protoplast pellet was embedded with 3 ml filter-sterilized (0.45 μM) alginate solution consisted of 1% w/v alginic acid sodium salt (A2158, Sigma) dissolved in Y3A liquid media (5.5% w/v sucrose and 11.9% w/v glucose supplemented with 10 μM NAA, 2 μM 2.4-D, 2 μM IBA, 2 μM GA3, 2 μM 2iP and 200 mg/L ascorbic acid) adjusted to pH 5.6, in which the Y3 macroelements was prepared without calcium chloride (CaCl₂.2H₂O).

Alginate Thin Layer Preparation

Alginate-embedded protoplasts were distributed as a thin layer onto supporting media comprising, 1.5 mL filter-sterilized Y3A (5.5% w/v sucrose and 11.9% w/v glucose supplemented with 0.1% w/v CaCl₂.2H₂O) solidified with 1% w/v agarose sea plaque, in 35 mm×10 mm petri dish (no. 627161, Greiner Bio-One, Germany). The distribution of alginate-embedded protoplasts was performed by dropping 100 μL alginate-embedded protoplasts at the edge of petri dish and immediately held the petri dish at an angel of 35° to allow the drop distributed as a thin layer. The dishes were placed horizontally into 94 mm×15 mm two compartment dishes (no. 635102, Greiner Bio-One, Germany) where the alginate solidified within 1-2 minutes. Three ml sterile water was added into the outer compartment in order to prevent the alginate layer from drying out. The plates were sealed and incubated at 28° C. in the dark for 3 days.

Microinjection Workstation

The microinjection workstation consisted of a Leica DM LFS upright microscope (Leica Microsystems Wetzlar GmbH, Germany) with a joystick controlled motorized objective revolver for HCX APOL U-V-I water immersion objectives (10×, 20×, 40× and 63×), mounted on a fixed table and placed in a laminar. The microscope was equipped with a Luigs and Neumann Manipulator set with a control system SM-5 and SM-6 (Luigs and Neumann, Germany).

Preparation of DNA Injection Solution

Plasmid DNA was prepared by midi scale Plasmid DNA Purification Kit (NucleoBond [Registered Trade Mark] PC 100; MACHEREY-NAGEL, Germany) and was dissolved at concentration of 1 μg/μl in sterile water. The plasmid was restricted with Hindrn and EcoRI to yield the CFDV-hrGFP-nos cassette as a 1.5 kb fragment. The fragment was separated from the vector sequence (pUC19) by electrophoresis on a 1% w/v agarose gel. The DNA fragment containing the cassette was excised using a clean blade and isolated using the PCR clean-up Gel extraction Kit (NucleoSpin [Registered Trade Mark] Gel and PCR Clean-up) according to the manufacturer's description (MACHEREY-NAGEL, Germany). The DNA cassette was then diluted with sterile water to concentrations of 100 ng/μl. The DNA solution was mixed with Lucifer Yellow CH dilithium salt (L0259, Invitrogen) in a proportion of 10:0.1 and filter-sterilized using the Ultafree-MC filter (Durapore 0.22 μm, type: GV; No. SK-1M-524-J8; Millipore) by spinning at 10,000 rpm, 15 minutes at 22° C. The eluted DNA were partitioned into 10 μL aliquots as DNA injection solution and stored at −20° C. until required.

Loading the DNA Injection Solution into Microinjection Needle

The DNA injection solution was centrifuged at 14,000 rpm for 30 minutes at 4° C. before loading into Femtotip II microinjection needle (no. 5242 957.000, Eppendorf). A 5 μL aliquot of DNA injection solution was loaded as close as possible to the tip of Femtotip II microinjection needle through back opening of the needle using microloader (no. 5242 956003, Eppendorf). After 30 minutes standing at room temperature, the needle was filled with sterile mineral oil (M8410, Sigma) using the microloader and tightly mounted in the capillary holder of microinjector CellTram vario (no. 5176 000.033, Eppendorf), and then fixed onto micromanipulator.

Microinjection of Oil Palm Protoplasts

A plate containing alginate layer protoplasts was placed on the microscope stage, and the vitality of embedded protoplasts was confirmed by using the 10× objective. The objective was raised to maximum position to freely allow the needle tip to reach the center of the field view with the X- and Y-axis controller (Control system SM-5) of the manipulator. The needle was lowered as close as possible to the alginate layer with the Z-axis controller and the cytoplasm or nucleus of target protoplast was identified by adjusting the 20× objective to optimal resolution and contrast, after which the needle tip was moved to right above the protoplast with the X- and Y-axis hand wheel controller. The needle tip was then inserted into the alginate layer just next to the protoplast by using Z-axis hand wheel controller and penetrated into the protoplast by using the X-axis hand wheel controller. The DNA injection solution was slowly injected into the protoplast by using a microinjector CellTram vario, which was confirmed by the fluorescence illumination. The needle tip was carefully withdrawn from the protoplast and moved to the next target protoplast. The injected protoplasts were monitor periodically by using Leica MZ16F fluorescent stereomicroscope with GFP3 filter (Leica Microsystems Wetzlar GmbH, Germany).

Alginate Layer Culture

Following microinjection, the plates containing the alginate layer were incubated in the dark at 28° C. for 5 days. The alginate layers were then separated from supporting media and transferred into 60 mm×15 mm petri dishes containing 3 mL Y3A liquid media consisted of 5.5% w/v sucrose and 8.2% w/v glucose supplemented with 10 μM NAA, 2 μM 2,4-D, 2 μM IBA, 2 μM GA3, 2 μM 2iP and 200 mg/L ascorbic acid. The plates were incubated in the dark by shaking at 50 rpm at 28° C. After 2 weeks, the media was replaced with similar Y3A liquid media but the concentrations of sucrose and glucose were decreased to 4% w/v and 7.2% w/v, respectively. The alginate layers were cultured in this media for a month by refreshing the media at 14-days intervals, then replaced with Y3A liquid media comprising of 4% w/v sucrose until the microcalli were observed.

### Example 1

### Protoplasts Isolation

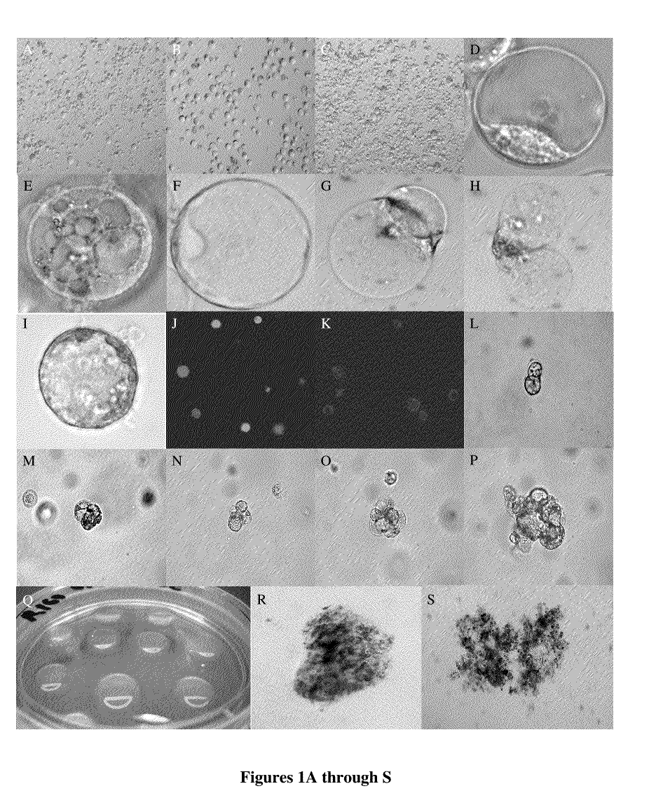

Protoplasts were successfully isolated from suspension cultures at 4, 7 and 14 days after subculture with yields of 0.9-1.14×10⁶ per g fwt and with an average viability of 82%. The sizes of the protoplasts were 5-14 μM, 15-25 μM and 25-35 μM, which were isolated from 4, 7 and 14 days after subculture, respectively (FIGS. 1A-C).

For oil palm, Bass and Hughes (1984) Plant Cell Rep 3:169-172 reported protoplast isolation from suspension cultures. Sambanthamurthi et al. (1996) Plant Cell, Tissue and Organ Culture 46:35-41, Srisawat and Kanchanapoom (2005) Science Asia 31:23-28 and Te-chato et al. (2005) J Sci Technol 27(4):685-691 reported protoplast isolation of embryogenic callus in solid media, in which only microcalli were produced. In accordance with the present disclosure, the protoplasts were successfully isolated from suspension culture with cell division to form microcolonies. The microcolonies successfully grew to microcalli and, for the first time, the microcalli regenerated to plants through the process of somatic embryogenesis.

The present isolation protocol is very efficient for protoplast isolation from oil palm suspension cultures since it is based on optimal centrifugation steps to obtain high yield and viability and is substantially free from contamination by cell debris. In the present protocol, protoplast yield was up to 1.14×10⁶ per g fwt of protoplasts.

In this Study, the yield and viability of the protoplasts obtained from 4 to 14 days after subculture did not show any significant difference. However, the size of protoplasts varied in the range of 5-35 μm. Protoplasts were isolated seven days after subculture for subsequent experiments as this gave an average size of 15-25 μM with the protoplasts showing dense cytoplasm concentrated around the nucleus which was more favorable to cell division besides being easier to handle and monitor (FIG. 1D). Whilst 25-35 μM protoplasts were also fully packed with cytoplasm, cell division capacity was reduced (FIG. 1E), and more than 50% of the protoplast less than 15 μM in size had cytoplasm without a nucleus (FIG. 1F).

In addition, protoplasts isolated at 7 days after subculture were in the exponential phase (Teixeira et al. (1995) supra), which enabled a high degree of cell division. The high degree of cytoplasmic activity during protoplast culture and the availability of space to grow inside the 15-25 μM protoplast likely contributed to the success of plant regeneration in this study. This was shown after three days where the volume of cytoplasm increased and eventually distributed by filling the whole cell before initial the cell division (FIGS. 1G&H).

In this study, plant regeneration was achieved by using protoplasts isolated from three month old suspension cultures. Protoplasts isolated from older than three months old suspension cultures were observed having large starch granules which ruptured easily in the isolation process resulting in a decrease in protoplast yield (FIG. 11). Furthermore, an attempt to culture using these protoplasts resulted in no cell division and generally 80% of the protoplasts died after 5-14 days of culture. In addition, when suspension culture was older than 3 months, it contained more compact cell clusters than friable cell clusters. Compact cell clusters were unable to undergo somatic embryogenesis.

### Example 2

### Selection of Optimum Media

In order to identify appropriate media for cultivation of oil palm protoplasts, 14 media combinations were compared, as shown in Table 2. The components of the media contributed to successful protoplast culture. EC version media (1/2ECI-ECVI) were prepared based on MS basal media (Murashige and Skoog (1962) Physiologia Plantarum 15:473-497) except for ECII, which was based on KM basal media (Kao and Michayluk, (1975) Planta 12:105-110). Meanwhile, Y3 version media (Y3-Y3F) were based on the modified Y3 basal media (Teixeira et al. (1995) supra). Other components of the media were included based on media used in other plant species.

Preliminary experiments indicated that when protoplasts were cultured in liquid medium, division was not observed in all media tested and extensive aggregation of protoplasts was observed. Most of the protoplasts died after two week of cultivation. In contrast, protoplasts embedded in solidified agarose remained viable (FIG. 1J) and showed cell wall formation (FIG. 1K) in cell division (FIG. 1L) in all media tested. Overall, Y3-Y3F media was highly superior in its ability to initiate cell wall formation (5-7 days), first cell division (9-14 days) and second cell division (17-21 days; FIG. 1M) of oil palm protoplasts (Table 4). The highest protoplast division frequency at 12% was recorded in Y3A media with cell wall and first cell division occurring at five days and nine days cultivation, respectively. This follows Y3D media with 8% of protoplast division frequency at 10 days. EC version media caused a lag time of 10-15 days prior for cell wall formation and the frequency of protoplast division was low (0.5-2.1%).

TABLE 4
Protoplasts division frequency in different media used

| | Days for Cell | Days for 1^{st} | Days for 2^{nd} | Div. |
|---|---|---|---|---|
| Media | wall formation | division | division | frequency |
| 1/2ECI | 15 | 21 | 28 | 0.49 ± 0.23 |
| ECI | 15 | 21 | 28 | 0.55 ± 0.19 |
| ECII | 10 | 17 | 23 | 1.77 ± 0.19 |
| ECIII | 12 | 17 | 25 | 1.10 ± 0.38 |
| ECIV | 13 | 19 | 24 | 1.11 ± 0.19 |
| ECV | 13 | 20 | 26 | 0.67 ± 0.58 |
| ECVI | 10 | 17 | 21 | 2.11 ± 0.19 |
| Y3 | 6 | 14 | 21 | 3.21 ± 0.5 |
| Y3A | 5 | 9 | 12 | 12 ± 0.88 |
| Y3B | 7 | 14 | 21 | 4 ± 0.33 |
| Y3C | 6 | 12 | 19 | 5.56 ± 0.19 |
| Y3D | 5 | 10 | 17 | 7.94 ± 0.58 |
| Y3E | 7 | 14 | 21 | 4.89 ± 0.38 |
| Y3F | 5 | 10 | 17 | 6.24 ± 0.19 |

The Y3 version media especially Y3A media was identified as the optimum media for protoplasts cultures in this study. This was the first time that the modified Y3 media was used for oil palm protoplasts culture. MS based media and AA media (EC version media) in this study was unsuccessful due to very low protoplasts division frequencies.

The components of macroelements and microelements in Y3 version media were similar to original Y3 media except the potassium dihydrogen phosphate (KH₂PO₄) was added in Y3A and Y3D˜Y3F media. Addition of KH₂PO₄ increased the protoplast division frequency compared to Y3 version media without KH₂PO₄ (Y3, Y3B, Y3C). In the isolation process, the protoplasts are exposed to stress and damage and when placed in the media, the adsorption of nutrients especially phosphate ions occurs intensively for the first 1-3 days to repair the damage cells (Chaillou and Chaussat (1986) Phytomorphy 36:263-270). Thus, the addition of KH₂PO₄ was required to balance the nutritional requirements in all Y3 version media.

The Y3 version media contained a higher concentration of chloride ions (Cl⁻) compared to the EC version media by the presence of ammonium chloride (NH₄Cl), potassium chloride (KCl) and nikel chloride (NiCl₂.6H₂O). Although the chloride ions are known to act like natural auxins in the induction of plant root formation, they may also play an important role in the growth of oil palm protoplasts. The presence of ammonium nitrate (NH₄NO₃) in the microelements of media has been assumed to prevent the cell division of protoplasts in many woody plants such as poplar (Qiao et al. (1998) Plant Cell Rep 17:201-205). In this study, protoplasts culture using media without NH₄NO₃ (ECVI media) did not show any adverse effect but the ratio of NH₄ and NO₃ influenced the division of protoplasts. High protoplast division frequencies were obtained from Y3 version media which containing 1:4 ratio of NH₄ and NO₃ (NH₄Cl and KNO₃) compared to 1:2 ratio (NH₄NO₃ and KNO₃) in EC version media.

### Example 3

### Selection of Optimum Plant Growth Regulators (PGRs)

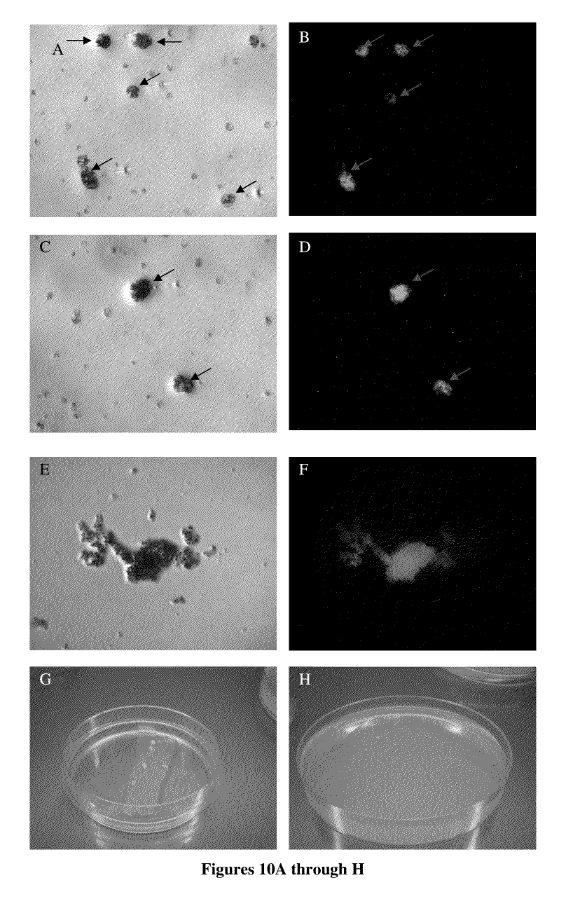

After 8 weeks cultivation of the divided protoplasts, the media became unable to promote the development of microcolonies (7-12 cells). The protoplasts were observed to divide into hardly more than five cells and half of them maintained 3-4 dividing cells (FIG. 1N). Therefore, the addition of plant growth regulators (PGRs) in used media was found to be essential and had to be optimized to induce the microcolony formation without inhibiting plant regeneration. Eleven combinations of PGRs (Table 3; PGRs No. 1-11) consisting of different concentrations of four auxins [NAA, 2,4-D, indole-3-acetic acid (IAA), indole-3-butyric acid (IBA)] and four cytokinins [zeatin (Zea), gibberellic acid A3 (GA3), BA, 2iP] were tested in order to regulate the growth of the protoplast cultures in Y3A and Y3D media. Table 5 showed that all concentrations of PGRs for combination nos. 1-6 completely inhibited the growth of protoplasts either in Y3A or Y3D. The concentration of 2,4-D, IBA, GA3 and 2iP higher than 7 μM inhibited cell division, and surprisingly, the protoplasts died within a week. The formation of microcolonies was observed in both media after 12-16 weeks at all concentrations of PGRs for combination nos. 9-11 (FIG. 10). The protoplasts cultured in Y3A media supplemented with the combination PGRs no. 10 at a concentration of 10 μM NAA, 2 μM 2,4-D, 10 μM, 2 μM IBA, 10 μM Zea, 2 μM GA3, 10 μM BA and 2 μM 2iP gave the highest division frequency at 18.33% and microcolony formation frequency at 8.86%. For Y3D media, the PGRs combination no. 10 was also able to promote 7.38% of dividing protoplasts to the 3.14% of microcolony formation in which the division frequency was slightly lower compared to 8.19% division frequency of Y3D media without PGRs.

TABLE 5
Effect of PGRs combination for protoplast
cultures using Y3A and Y3D

| | agarose solidified culture | | | |
|---|---|---|---|---|
| | Y3A | | Y3D | |
| | Div. | Microcolony | Div. | Microcolony |
| PGRs No. | frequency | frequency | frequency | frequency |
| Without | 11.16 ± 0.9 | — | 8.19 ± 1.34 | — |
| PGR | | | | |
| 1 | — | — | — | — |
| 2 | — | — | — | — |
| 3 | — | — | — | — |
| 4 | — | — | — | — |
| 5 | — | — | — | — |
| 6 | — | — | — | — |
| 7 | 3.51 ± 1.09 | 1.82 ± 0.46 | 0.53 ± 0.92 | |
| 8 | 6.64 ± 2.24 | 2.98 ± 0.61 | 1.76 ± 1.54 | — |
| 9 | 9.25 ± 1.01 | 4.40 ± 0.78 | 4.64 ± 1.06 | 0.73 ± 0.66 |
| 10 | 18.33 ± 0.44 | 8.86 ± 0.85 | 7.38 ± 1.16 | 3.14 ± 0.65 |
| 11 | 14.37 ± 1.83 | 6.39 ± 0.93 | 6.29 ± 2.33 | 1.46 ± 0.62 |

| | Y3A | |
|---|---|---|
| | Microcolony frequency | Microcalli frequency |
| 10 (control) | 7.76 ± 0.38 | 0 |
| 12 | 11.3 ± 2.42 | 1.6 ± 0.7 |
| 13 | 11.9 ± 1.55 | 5.5 ± 2.13 |
| 14 | 3.56 ± 2.64 | 0 |
| 15 | 13.06 ± 2.95 | 6.13 ± 1.58 |
| 16 | 6.6 ± 0.96 | 0 |
| 17 | 5.03 ± 1.95 | 2.73 ± 2.12 |
| 18 | 4.53 ± 1.65 | 0 |
| | Agarose beads culture | |
| | Microcolony frequency | Microcalli frequency |
| 19 | 22.2 ± 4.75 | 9.8 ± 1.87 |

In order to identify the effects of each hormone in the PGRs combination no. 10 for the development of microcolonies into microcalli (15-30 cells), seven combinations of PGRs were evaluated in Y3A media by excluding one of the hormones in each combination as shown in Table 3 (PGRs nos. 12-18). Among the PGRs combinations tested, the numbers of microcolonies were significantly increased at frequencies of 11.3%, 11.9% and 13% when cultured with PGRs combination nos. 12, 13 and 15 in which 2 μM 2,4-D, 10 μM IAA and 10 μM Zea were excluded (Table 5). In contrast, when excluding the concentration 2 μM of each IBA, GA3 and 2iP in PGRs combination no. 14 and 16-18, the microcolony frequencies were decreased to range between 3.56% and 5.03%. The microcolonies developed further into microcalli (FIG. 1P) and appeared visible to the naked eye after 16-20 weeks cultures in Y3A media with PGRs combinations nos. 12, 13, 15 and 17, in which the highest microcalli formation frequency of 6.13% was obtained from PGRs combination no. 15 and the lowest of 1.6% from the combination no. 12. The absence of IBA, GA3 and 2iP in PGRs combinations nos. 14, 16 and 18 adversely influenced the formation of microcolonies and no microcalli could be developed even when culture was extended to 20 weeks.

This study determined the optimum PGRs for successful plant regeneration of oil palm protoplasts. PGR concentrations and combinations need to be optimized for protoplast development into plants. In this study, the continuous growth of the protoplasts was clearly affected by the combination of PGRs in each step. Efficient protoplast development to microcalli was obtained when the Y3A media was supplemented with 3 auxins (10 μM NAA, 2 μM 2,4-D, 2 μM IBA) and 2 cytokinins (2 μM GA3, 2 μM 2iP).

The low concentration of IBA, GA3 and 2iP was essential for oil palm protoplast culture as no microcalli were observed whenever these PGRs were excluded from Y3A media. The division frequencies obtained were lower when the protoplasts were cultured in media supplemented with higher than 2 μM concentration of 2,4-D, IBA, GA3 and 2iP compared to without PGRs. Two μM each of 2,4-D, IBA, GA3 and 2iP was identified as optimum concentration for the development of protoplasts to microcalli since the frequency of cell division and formation of microcolonies and microcalli was substantially reduced when 1 μM of these PGRs was used.

### Example 4

### Effects of Osmotic Pressure, Optimum PGRs using Agarose Beads Culture

Based on the results of PGR optimization, the protoplasts were most efficaciously cultured using the agarose beads technique (FIG. 1Q) comprising Y3A media supplemented with 10 μM NAA, 2 μM 2,4-D, 2 μM IBA, 2 μM GA3 and 2 μM 2iP which was designated as PGRs combination nos. 19 (Table 3). The agarose beads were cultured for three days by surrounding the beads with 21% osmotic solution of either sucrose, glucose or mannitol to maintain the osmotic pressure and to prevent the agarose beads from drying out. The use of different types of carbohydrate as the osmotic solution did not adversely effect the protoplast cultures. However, the protoplasts cultured in the osmotic solution were observed to retain a sphere shape and viability compared to those cultured in the absence of osmotic solution where the protoplasts became oval shaped and half of them burst and died. Higher than 21% osmotic solution quickly changed the agarose beads to a brown color and lead to the formation of pyramid likes crystal on the surface of the agarose beads.

After three days, the osmotic solution was replaced with Y3A liquid media (Table 2) without PGRs. Longer than five days in the osmotic solution resulted in protoplasts becoming a dark color and they developed fur-like structures on the surface of cell wall which retarded cell division. Culturing the agarose beads in Y3A liquid media promoted the cell division and development of microcolonies in eight weeks at the frequency of 22.2%, which was significantly higher than PGRs combination no. 15 where the frequency was 13%. At this stage, the agarose beads were cultured in Y3 liquid media consisting of 4.5% w/v sucrose and resulted in the development of microcalli at the frequency of 9.8% at compared to 4.13% of PGRs combination no. 14. Besides IAA, Zea and BA were excluded from PGRs combination no: 18 and the osmotic pressure surrounding the agarose beads was gradually reduced which increased the numbers of microcolonies and microcalli by two fold.

The use of suspension cultures, optimum media and PGRs alone did not lead to the successful regeneration of plants in this study. Previous studies showed that the development of embryogenic calli from microcalli was a critical problem for oil palm protoplasts cultures. The use of agarose bead culture was identified as one of the factors in which highest frequency of formation of microcolonies (22%) and then further development to microcalli (9.8%) compared to protoplasts embedded in agarose solidified culture. Both culture techniques could protect and maintain the protoplast, agarose bead cultures which allowed for easy transfer and there was minimal disturbance of the protoplasts. Furthermore, the entire agarose bead was in direct contact with liquid media compared to solid media in which only the bottom part of an agarose bead was in contact with the media.

The used of liquid media with different osmotic pressures surrounding the agarose beads was identified as another factor which enhanced the development of embryogenic calli. The time points selected to change these media also influenced the growth of microcalli to embryogenic calli. Earlier or later the time points, the more retarded the growth of protoplasts. In early protoplast culture (3 days), high osmotic pressure surrounding the agarose beads was maintained by using 21% w/v carbohydrate solution which was then slightly reduced by Y3A liquid media consisting of 4% w/v sucrose and 7.2% w/v glucose at day 4, and reduced to normal osmotic pressure by Y3 liquid media consisting of 4.5% w/v sucrose when microcalli were observed at weeks 24.

### Example 5

### Control of Callus Browning

After 28 weeks, the microcalli failed to further grow to embryogenic calli. It was observed that the microcalli turned brown and light-dark due to the accumulation of phenolic compounds released from the cells and also the chemical reduction of PGRs in the agarose beads. Adding ascorbic acid (AA), silver nitrate (AgNO3) or activated charcoal (AC) with PGRs to the surrounding media of agarose beads reduced the microcalli browning process and promoted embryogenesis. The agarose beads were cultured in Y35N5D2iP liquid media with the addition of different concentrations of AA, AgNO3 and AC. After 4 weeks of cultivation the microcalli become yellowish and then developed embryogenic callus, indicating a further growth of the cells, especially when cultured in media containing 200 mg/l of AA. In comparison, culturing the agarose beads in Y3 liquid media with 200 mg/l AA without PGRs resulted in fewer embryogenic calli forming.

The chemical reduction of PGRs and the accumulation of phenolic compounds in the agarose beads were identified as the reasons that led the microcalli browning which retarded the growth of microcalli to embryogenic calli. The use of Y35N5D2iP liquid media supplemented with 200 mg/l ascorbid acid resulted in the development of embryogenic callus from microcalli. In contrast, the used Y35N5D2iP supplemented with AgNO3 or AC did not solve the problem of browning and surprisingly the microcalli browning became worst. This could be due to AgNO3 being more effective in adsorbing ethylene than the phenolic compounds. In contrast, AC adsorbed not only the phenolic compounds but also PGRs or vitamins from the media (Davey et al. (2005) Biotanicol Adv 23:131-171).

### Example 6

### Plant Regeneration

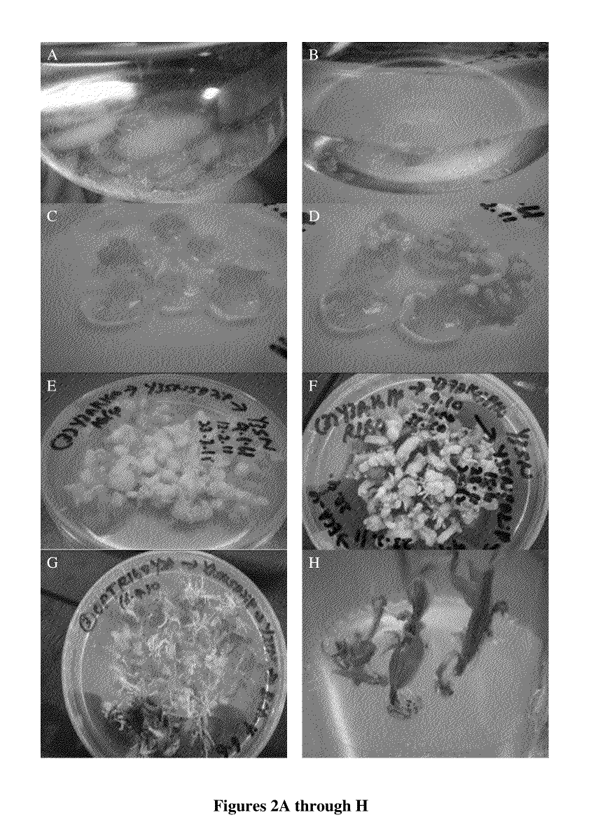

Eight weeks after culture initiation in Y35N5D2iP liquid media with the addition of 200 mg/l AA, two types of embryogenic callus developed as compact embryogenic (CE) callus (FIG. 1R) and friable embryogenic (FE) callus (FIG. 1S) with some of the embryogenic callus developing out from the agarose beads (FIGS. 2A and B). At this time, the agarose beads were transferred onto Y3 liquid media supplemented with different concentration of PGRs (NAA and BA) to promote the embryogenic calli to enter the somatic embryogenesis stage. Of five different concentrations of PGRs (NAA and BA) tested, only Y3 liquid medium supplemented with 1 μM NAA and 0.1 μM BA (Y31N0.1BA) was able to induce the FE embryogenic calli to develop into somatic embryos (FIG. 2C). In contrast, the CE callus was observed during the development of FE callus prior to the somatic embryogenesis stage. The agarose beads were subcultured in four-week intervals on Y31N0.1BA solid media until all the embryogenic calli were developed to somatic embryos (FIG. 2D). After 36 weeks of agarose bead culture, whitenish embryoids (FIG. 2E) appeared on the surface of agarose beads which were transferred onto ECI solid media with 1 μM NAA and 0.1 μM BA (ECI1N0.1BA). The greenish embryoids (FIG. 2F) were observed within eight weeks when cultured in the presence of light and regenerated into plantlets in another 12 weeks (FIGS. 2G and H).

Plant regeneration from protoplast-derived embryogenic callus was greatly influenced by media supplemented with low concentration of NAA and BA. Somatic embryogenesis was only observed when the agarose beads were cultured on Y31N0.1BA solid media. Y35N5D2iP liquid medium is preferably changed to Y31N0.1BA solid medium as soon as embryogenic callus observed. Longer cultivation in Y35N5D2iP liquid media retained the growth of embryogenic callus in callusing stage which delays the plant regeneration process. Furthermore, more CE callus was developed compared to FE callus which showed more callus multiplication than callus proliferation. Plant regeneration from protoplast-derived somatic embryos showed a similar growth pattern of plant regeneration from embryogenic callus cultures. Most of the embryos developed into normal small plantlets after subculture onto ECIIN0.1BA.

Nearly 14 months after protoplasts were isolated, true plants were generated using agarose bead culture. The protoplasts developed to microcolonies in eight weeks, to microcalli in 24 weeks and to small plantlets in 56 weeks under the culture conditions described. Further improvement to accelerate regeneration contemplated herein include protoplast culture using alginate layer technique, heat shock treatments prior to protoplast culture and the addition of ascorbic acid in the media throughout the process.

### Example 7

### Transient Expression of PEG-Mediated Oil Palm Protoplasts Transformation

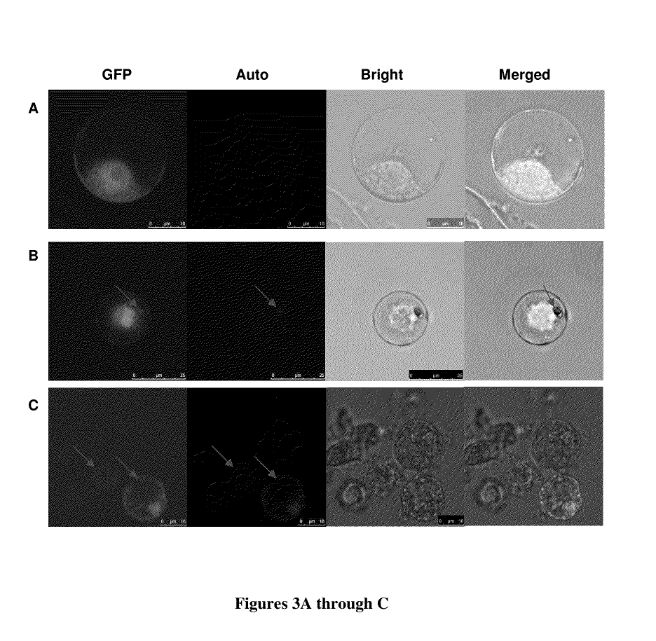

In order to identify the most suitable protoplasts for DNA uptake using PEG-mediated transformation, different sources of protoplasts were used defined as 7 days and 14 days after subculture of 3 month old embryogenic suspension culture, or 4 month old suspension culture. Initial protoplast transfection experiments used 10 μg of CFDV-hrGFP plasmid DNA (coconut foliar decay virus promoter operably linked to DNA encoding humanized renilla green fluorescent protein), incubation for 10 minutes and mixing with 40% w/v PEG dissolved in Rinse solution. The presence of green fluorescing protoplasts indicated expression of the hrGFP gene and these were detected at 72 hours after PEG-mediated transformation for all sources of protoplasts (FIG. 3). However, only low transfection efficiencies (<0.1%) were achieved in which green fluorescence was only observed only in viable (i.e. not ruptured) protoplasts. Protoplasts isolated from 7 and 14 days subcultures showed green fluorescence localized throughout the cytoplasm and nucleus extending to the plasma membrane (FIGS. 3A and B), whilst, green fluorescence was distributed throughout the whole cell for protoplasts from 4 month old suspension culture (FIG. 3C). However, weak and high intensity autofluorescences were detected in protoplasts from 14 day subculture of 3 month old suspension culture and 4 month old suspension culture, respectively. The protoplasts isolated from the suspension culture should not have chloroplasts or chlorophyll, thus the autofluorescences could be due to the presence of the small amount of lipids inside protoplasts from both sources, which showed pale yellow fluorescence in merged images (FIGS. 3B and C). Studies from Sambanthamurthi et al. (1996) supra showed that osmotic stress during protoplasts isolation probably induced the alteration of lipid metabolism resulting in the synthesizing of up to about 27% palmitoleic acid. Thus, the protoplasts isolated from 7 day subculture of 3 month old suspension culture were the most suitable for PEG-mediated transformation due to no autofluorescences which would give a false green fluorescence of hrGFP gene expression. Furthermore, the protoplasts were highly uniform in size and transfected protoplasts more easily identified and regenerated into plants.

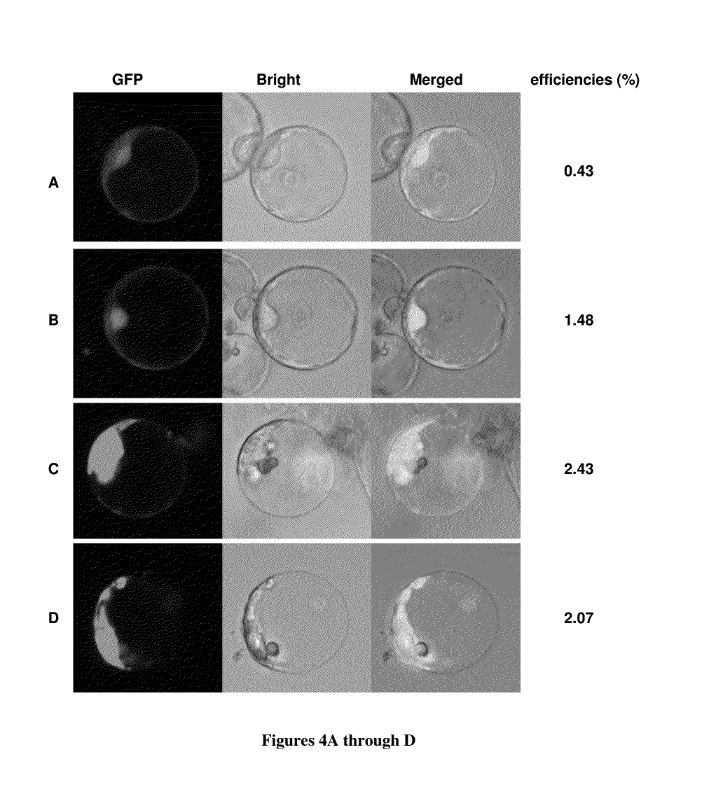

Ion exchanged during PEG-mediated transformation of the protoplasts greatly influenced the transfection efficiency and intensity of green fluorescence following hrGFP gene expression. The protoplasts was exposed to Ca²⁺ ions by incubation in Washing solution comprising of CaCl.2H₂O followed by exposure to Mg²⁺ ions using PEG-MgCl₂ solution and then again to Ca²⁺ ions when the protoplasts-PEG solution was diluted with Washing solution. To examine the effects of Mg²⁺ ions on transfection efficiency, oil palm protoplasts were incubated for 10 minutes with 10 μg of CFDV-hrGFP plasmid DNA and mixed with 40% w/v PEG dissolved in Rinse solution comprising 10 mM, 25 mM, 50 mM and 100 mM of MgCl₂.6H₂O. Addition of 10 mM MgCl₂.6H₂O in PEG solution resulted in drastically increased the transfection efficiency by 4 folds (0.43%, FIG. 4A) compared to without MgCl₂.6H₂O (<0.1%). The transfection efficiencies were consistently increased to 2.43% by the increasing of MgCl₂.6H₂O concentrations as shown in FIGS. 4B through D. Furthermore, the expression of the hrGFP gene was highly and consistently influenced by the presence of Mg²⁺ ions as indicated from low to high intensity of green fluorescence (FIGS. 4A through D).

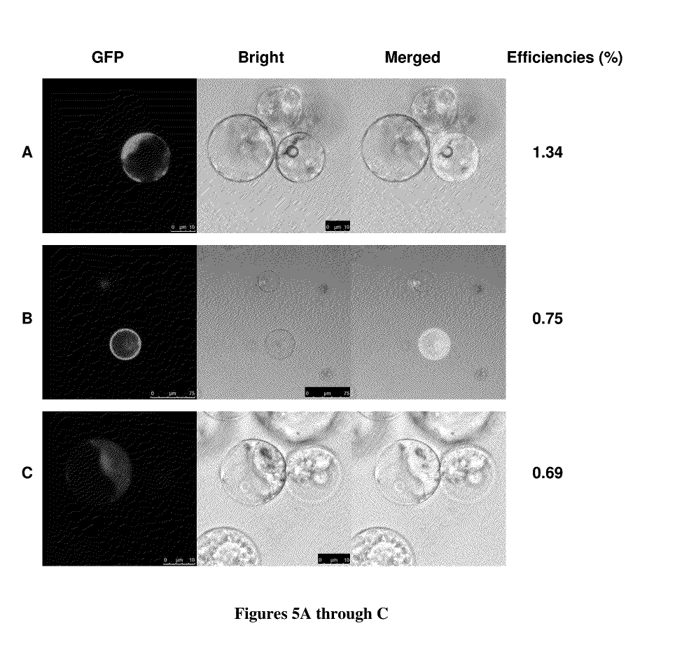

Transformation of the protoplasts was tested using longer DNA incubation times. However, when DNA incubation time was prolonged to 15 or 30 minutes before addition of PEG-MgCl₂ solution (FIGS. 5A and B), the transfection efficiency decreased to 0.75%. The results showed that PEG-MgCl₂ solution added after 10 minutes or less of DNA incubation likely reduced excretion or activity of DNases from the protoplasts or cellular nucleases. Addition of carrier DNA in the form of 50 μg of sonicated salmon sperm DNA mixed with 25 μg of CFDV-hrGFP plasmid DNA for 30 minutes reduced transfection efficiency to 0.69%. This suggests the inhibition of the ability of the plasmid DNA to permeabilize the protoplast membrane (FIG. 5C).

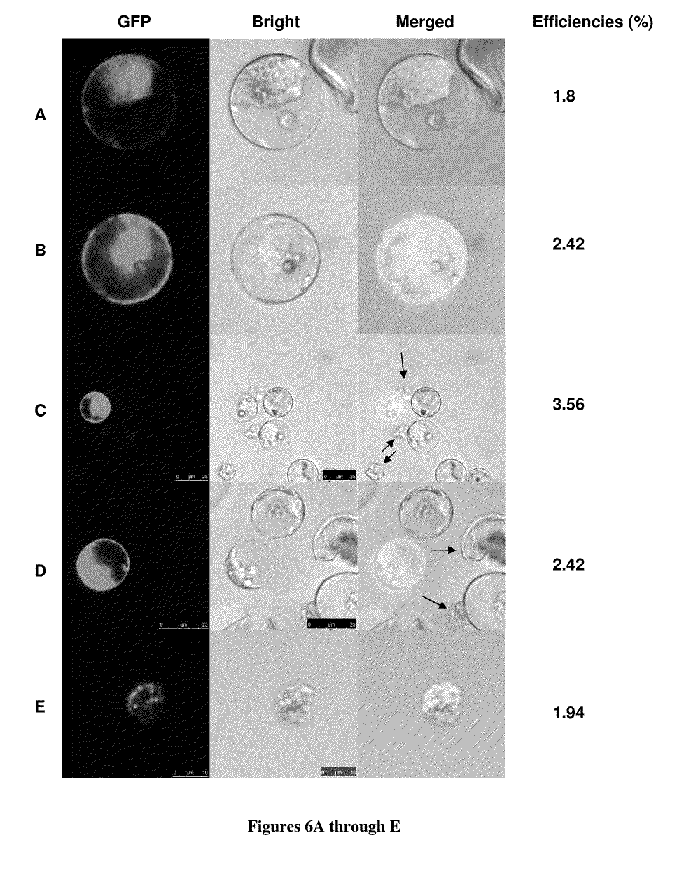

To investigate the effects of the amount of DNA introduced into oil palm protoplasts on transfection efficiency, 25 μg and 50 μg of CFDV-hrGFP plasmid DNA was transfected into oil palm protoplasts by using PEG-MgCl₂ solution (40% w/v/ PEG and 50 mM MgCl₂.6H₂O). The results showed transfection efficiencies of 1.8% for 25 μg which increased to 2.42% for 50 μg DNA (FIGS. 6A and B). Based on the intensity of green fluorescence, high level of hrGFP gene expression was observed at both concentrations of plasmid DNA. Green fluorescence concentrated in the cytoplasm for 25 μg of plasmid DNA and green fluorescence distributed over the whole cell of protoplasts for 50 μg of plasmid DNA. Hence, optimal concentrations of DNA provide the greatest transformation efficacy in PEG-mediated transformation.

PEG at a molecular weight of 4000 was selected to optimize the effect of PEG concentration on transfection efficiency of oil palm protoplasts. PEG concentrations at 25% w/v, 40% w/v and 50% w/v were used to transfect 50 μg of CFDV-hrGFP plasmid DNA into oil palm protoplasts which resulted in the transfection efficiencies of 3.55%, 2.42% and 1.95%, respectively (FIGS. 6C through E). The data show that 25% w/v PEG concentration was the optimal concentration for PEG-mediated transformation of oil palm protoplasts. The intensity of green fluorescence was at the same level for all concentration of PEG indicating that hrGFP gene expression was not influenced by PEG concentration. The toxicity of PEG caused the viability of the oil palm protoplasts to reduce to 30-50% when higher than 25% w/v PEG concentration was used. The damaged protoplasts were observed surrounding the green fluorescing (viable) protoplasts which indicated that the oil palm protoplasts were very sensitive to the toxicity of PEG. The green fluorescing damaged protoplasts were also be observed when 40% w/v or 50% w/v PEG concentration was used indicating higher transfection efficiency could be achieved if oil palm protoplasts could withstand the toxicity of PEG.

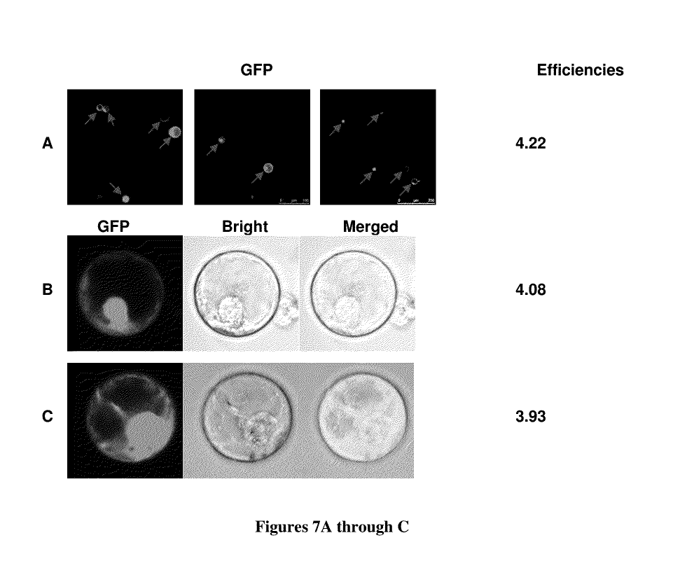

The effect of heat shock treatment was tested using the above optimized protocol. Oil palm protoplasts was treated by incubation at 45° C. for 5 minutes and placed on ice for 1 minute follows 10 minute incubation with 50 μg of CFDV-hrGFP plasmid DNA and then mixed with 25% w/v PEG solution consisting 50 mM MgCl₂.2H₂O. FIG. 7A shows transfection efficiency was further increased to 4.22% when heat shock treatment was incorporated with the optimized protocol. It is unclear why heat shock treatment influenced the PEG-mediated transformation of oil palm protoplasts and it could be that the plasma membrane of the protoplasts was altered when incubated at 45° C. allowing for greater DNA uptake. The green fluorescing protoplasts were observed continuously for 9 days indicated that hrGFP gene expression was retained with a less decrease in transfection efficiency, 4.08% at days 6 and 3.93% at days 9, without being interfered by the Washing solution (FIGS. 7B and C).

Stable Expression of DNA Oil Palm Protoplasts Mediated by DNA Microinjection

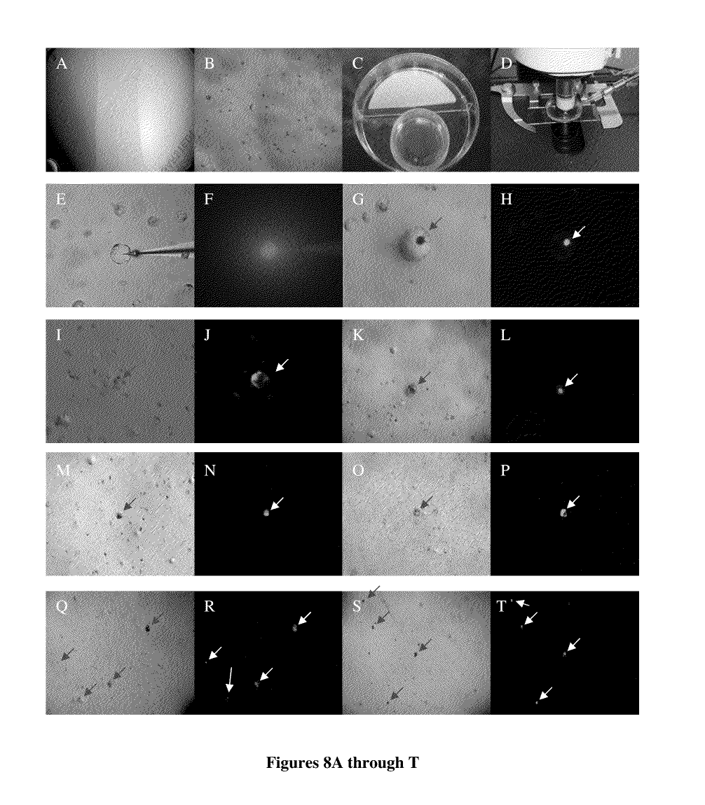

An attempt to inject protoplasts isolated from 7 day subculture of 3 month old suspension culture embedded in agarose bead was successful but only 5 to 10 cells can be injected within an hour due to the curve surface of the agarose beads resulting in difficult penetration of a needle tip at an angel of 35°. The target protoplasts were not easy to identify due to the position of the protoplasts at different layer. Furthermore, the needle tips were frequently clogged or broken after only 2-3 injections probably due to agarose particles accidentally blocked the needles tip. Thus, alginate layer embedded-oil palm protoplasts (FIG. 8A) were used for DNA microinjection since the protoplasts are in a single planar position (FIG. 8B). The transparent color of alginate makes it ideal for identification of the target protoplasts and microinjection can be performed on the next target protoplast in a shorter time period due to the flat surface of the alginate layer. Various concentrations of alginate (0.5-2% w/v) were dissolved in Y3A liquid media (5.5% w/v sucrose and 11.9% w/v glucose supplemented with 10 μM NAA, 2 μM 2,4-D, 2 μM IBA, 2 μM GA3, 2 μM 2iP and 200 mg/L ascorbic acid) and were used to embed the oil palm protoplasts for DNA microinjection. As a result, 1% w/v alginate was the optimal concentration to firmly fix the protoplasts in one plane which made it easier to facilitate injection. In contrast, lower and higher than 1% w/v alginate resulted in the moveable and accumulation of protoplast clumps, respectively.

Alginate layer-embedded protoplasts were cultured for 3-4 days in a two compartment dish (FIG. 8C) for partial development of the cell wall which was an optimal time for DNA microinjection. Freshly embedded protoplasts were damaged when the needle tip touched the plasma membrane demonstrating that the fragile membrane alone is sufficiently not hard enough to withstand the penetration of the needle tip. Meanwhile, DNA microinjection using protoplasts after 5 days of culture were difficult due to the cell wall being fully developed. Only one micromanipulator was used to inject the protoplasts because the protoplasts were firmly fixed inside the alginate layer (FIG. 8D).

Lucifer Yellow dye was essential as guidance for monitoring the DNA injection solution inside the target compartment of oil palm protoplasts (FIGS. 8E and F). Two compartments, nucleus (FIGS. 8G and H) and cytoplasm (FIGS. 8I and J), were successfully injected using a DNA fragment of CFDV-hrGFP. hrGFP gene expression in both compartments was only detected at 72 hours after DNA microinjection where green fluorescence was localized in the nucleus and cytoplasm as before. No green fluorescence was observed in the protoplasts injected with only Lucifer Yellow dye demonstrating the fluorescing nucleus and cytoplasm were from the expression of hrGFP gene. It was found that the Lucifer Yellow dye could maintain the fluorescence property for only 48 hours at 28° C.

hrGFP gene expression in the nucleus of oil palm protoplasts was detected up to 9 days of cultivation (FIGS. 8K and L) and disappeared at day 14 demonstrating that the nucleus was unsuitable for DNA microinjection. In contrast, the volume cytoplasm expressing the hrGFP gene was increased even at day 9 as shown in FIGS. 8M and N. Initial cell division was observed after 12 days (FIGS. 8O and P) and divided to 2 and 3 cells at days 21 (FIGS. 8Q and R), and then further developed to 4-6 cells in a month (FIGS. 8S and T). Fifty to 100 1% w/v alginated-embedded protoplasts were successfully injected within an hour using the optimal DNA microinjection procedure.

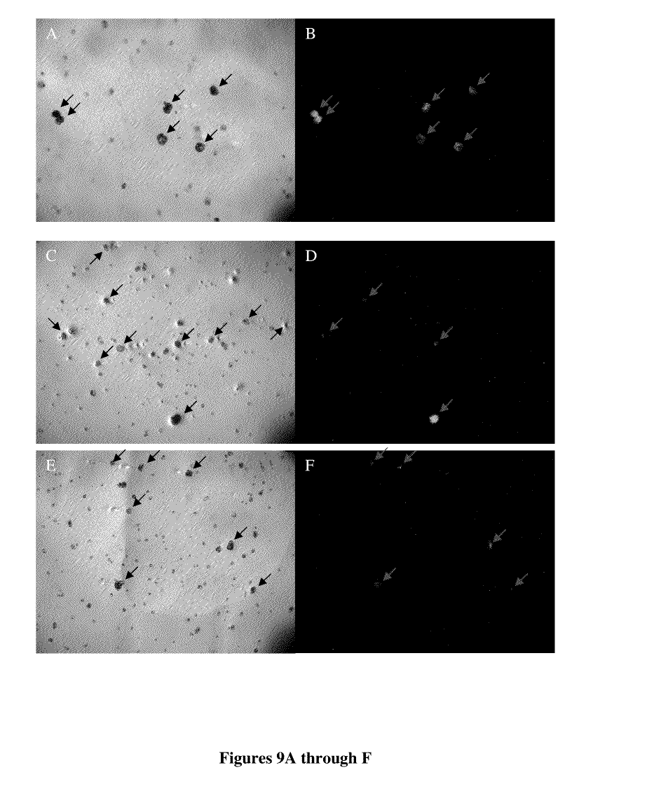

The optimal DNA fragment concentration was determined by DNA microinjection with three different concentrations, 100 ng/μL, 500 ng/μL and 1000 ng/μL of DNA injection solution. Fifty cells in the alginate layer-embedded protoplasts were injected with each concentration of DNA. After a month, 78% (39/50), 40% (20/50) and 10% (5/50) of transformation efficiencies were obtained from the protoplasts injected with 100 ng/μL (FIG. 9A), 500ng/μL (FIG. 9B) and 1000 ng/μL (FIG. 9C), respectively. The development of microcolonies which were injected with the optimal DNA concentration (100 ng/μL) were observed in 2 months but the transformation efficiency was decreased to 34% (17/50) (FIGS. 10A and B). The microcolonies maintained the expression hrGFP gene for another 2 months (FIGS. 10C and D) and decreased to 10% of transformation efficiency (5/50) when the microcalli were developed in 6 months (FIGS. 10E and F). The microcalli expressing hrGFP were removed from the alginate layer (FIG. 10G) and were transferred to Y31N0.1BA solid media (FIG. 10H) for further development of embryogenic callus which was similar to plant regeneration of protoplasts using agarose beads culture.

Those skilled in the art will appreciate that aspects of aspects described herein are susceptible to variations and modifications other than those specifically described. It is to be understood that these aspects include all such variations and modifications. These aspects also include all of the steps, features, compositions and compounds referred to or indicated in this specification, individually or collectively, and any and all combinations of any two or more of the steps or features.

What is claimed is:

1-32. (canceled)

33. A method for regenerating a plant of the genus Elaeis from a protoplast, said method comprising isolating the protoplast from a cell of an embryogenic suspension culture and culturing the protoplast in a growth medium supplemented with selected plant growth regulators comprising auxins and cytokinins and a source of phosphorous and potassium for a time and under conditions sufficient for microcallus to form from a microcolony of the cultured protoplasts and then regenerating a plantlet from the microcalli on solid media.

34. The method of claim 33, wherein the source of phosphorous and potassium is potassium dihydrogen phosphate.

35. The method of claim 33, wherein the growth medium is a Y3-based growth medium.

36. The method of claim 33, wherein the selected plant growth regulators comprise the auxin indole-3-butyric acid (IBA), the cytokinins gibberellic acid A3 (GA3), 2-γ-dimethylallylaminopurine (2iP), auxin naphthalene acetic acid (NAA), indole-3-acetic acid (IAA), the cytokinins zeatin (Zea), g-benzylaminopurine (BA) or an equivalent of any one or more thereof at a concentration of from about 1 μM to about 20 μM.

37. The method of claim 36, wherein the selected plant growth regulators comprise the auxins 10 μM NAA, 2 μM 2,4-D and 2 μM IBA, at least 2 μM of auxin IAA and at least 2 μM of cytokinin Zea, the cytokinins 2 μM GA3 and 2 μM 2iP.

38. The method of claim 33, wherein the protoplasts are cultured in an embedded solid phase.

39. The method of claim 33, wherein a nucleic acid is introduced, to modify protoplast, by polyethylene glycol (PEG)-mediated transformation of a suspension of protoplasts or by microinjection of nucleic acid in protoplasts embedded in a layer of gelatinous polysaccharide.

40. The method of claim 39, wherein from about 0.5 ng/μL to 2 ng/μL of nucleic acid is microinjected into the cytoplasm of the protoplast.

41. The method of claim 33, wherein the plant is selected from Elais guineensis, Elaeis oleifera (Elaeis melanococca) and Elaeis occidentalis.

42. A genetically modified plant of the genus Elaeis when regenerated from a genetically manipulated protoplast according to claim 41 or progeny or related generation of that plant which exhibits the genetic modification or a plant part thereof which comprise cells which express the genetic modification.

43. The genetically modified plant or plant part of claim 42 which produces a modified palm oil or palm kernel oil as a result of the genetic modification.

44. The genetically modified plant or plant part of claim 42 which produces a product resulting from the genetic modification.

45. The genetically modified plant or plant part of claim 42, wherein the product is a metabolite.

46. The genetically modified plant or plant part of claim 42, wherein the plant part comprises a leaf, root, stem, seed or a reproductive organ.

47. The method of claim 39, wherein the PEG-salt is 25% w/v PEG 4000 and 50 mM MgCl₂.6H₂O.
